(19) United States
(12) Patent Application Publication
(10) Pub. No.: US 2024/0117061 A1
(43) Pub. Date: Apr. 11, 2024

(54) ANTI-BCMA ANTIBODY, PREPARATION METHOD THEREFOR AND APPLICATION THEREOF

(71) Applicant: WUHAN YZY BIOPHARMA CO., LTD., Wuhan, Hubei (CN)

(72) Inventors: Lijuan FANG, Wuhan, Hubei (CN); Jian SHI, Wuhan, Hubei (CN); Shan HUA, Wuhan, Hubei (CN); Jing ZHANG, Wuhan, Hubei (CN); Kaili ZHANG, Wuhan, Hubei (CN); Pengfei ZHOU, Wuhan, Hubei (CN)

(21) Appl. No.: 18/257,749

(22) PCT Filed: Dec. 16, 2020
(86) PCT No.: PCT/CN2020/136748

§ 371 Date: Jun. 15, 2023

(51) Int. Cl. C07K 16/28; A61P 35/00

(52) U.S. Cl. CPC C07K 16/2878; A61P 35/00; C07K 2317/24; C07K 2317/31; C07K 2317/565

(57) ABSTRACT

An anti-BCMA antibody or an antigen-binding fragment thereof, and an application thereof in the treatment or prevention of tumor.

### TECHNICAL FIELD

The present invention relates to the field of pharmaceuticals, and particularly relates to an antibody against the B-cell maturation antigen (BCMA), a method for preparing the same and use thereof.

### BACKGROUND

Multiple Myeloma is abbreviated as MM. The development and progression of MM are multi-step processes. It starts with monoclonal gammopathy of undetermined significance (MGUS), and gradually progresses to smoldering MM (SMM), active MM and plasma cell leukemia (PCL). Statistically, the disease causes 114,000 new onsets and 80,000 deaths per year in the world, and the morbidity is still ascending year by year. This disease occurs more frequently among people aged 65-74 and the complications include hypercalcemia, renal insufficiency, anemia and infection. Currently, primary treatments for MM include autologous stem cell transplantation, in combination with conventional chemotherapeutics, immunomodulators (including thalidomide, lenalidomide and pomalidomide) and proteasome inhibitors (including bortezomib, carfilzomib and ixazomib). Although previously untreated MM patients are sensitive to existing therapies, MM may easily recur and develop drug resistance in the later stage of treatment. Therefore, there is an urgent need for a new treatment strategy for MM patients with relapse or metastasis to improve the current treatment situation.

The advent of immunotherapy has changed the therapeutic pattern for MM. Therapeutic monoclonal antibodies can effectively bind to effector cells expressing Fcγ receptor through the specialized Fc, and can induce the effector cells to generate antibody-dependent cell cytotoxicity (ADCC) effect, complement-dependent cytotoxicity (CDC) effect and antibody-dependent cell-mediated endocytosis to eliminate MM cells. In 2015, the U.S. FDA approved 2 therapeutic monoclonal antibodies, daratumumab targeting CD38 and elotuzumab targeting SLAMF7. Because of their low toxicity, the application of combination therapies of the antibodies with existing therapies to previously untreated MM patients and RRMM patients demonstrated significant clinical benefits. However, since the two antigens are expressed in normal activated B lymphocytes, T lymphocytes, mononuclear cells, natural killer cells (NK cells) and other effector cells, eliminating MM cells will reduce the number of associated effector cells during monoclonal antibody treatment, thereby reducing the killing effect of the monoclonal antibodies on MM cells. Meanwhile, since eliminating normal target cells has potential risks of non-specific killing, CD38 and SLAMF7 are not optimal therapeutic targets for MM.

BCMA, or tumor necrosis factor receptor superfamily member 17, abbreviated as TNFRSF17 (UniProt Q02223), is also known as B-cell maturation antigen (BCMA) or CD269. It is a non-glycosylated type III transmembrane protein that is widely expressed in plasmablasts and plasmocytes. Compared with the other two TNFRs, BAFF-R (B-cell activation factor receptor) and TACI (transmembrane activator and calcium modulator and cyclophilin ligand interactor), BCMA can participate in the transformation of late-stage memory B cells into PC cells. Meanwhile, in mice with BCMA knockout, it has been found that loss of BCMA only damages the survival of long-term PC, but does not affect early humoral immunity, the development and maturation of B cells and short-term immunoglobulin secretion.

Compared with the targets CD38 and SLAMF7, BCMA is specifically and highly expressed in MM tissues and cells and is associated with the progression of MM. BAFF-R is seldom detected in MM cell lines or MM cells of the patients. The positive rate and expression level of TACI are weaker than those of BCMA. In in vitro and in vivo studies on the biological functions of BCMA, the overexpression of BCMA can activate the downstream AKT, MAPK and NFκB signaling pathways, and induce the expression of key anti-apoptotic proteins Mcl1, Bcl2, Bcl-xL, pro-angiogenic protein CD31 and vascular endothelial growth factor (VEGF). The overexpression of BCMA can also regulate the activation, adhesion, angiogenesis and transfer of osteoclasts, and promote the expression or secretion of immunosuppression-related proteins and cytokines in osteoclasts, such as PD-L1 (programmed death ligand 1), TGF β (transforming growth factor β) and IL-10 (interleukin 10). Thus, blocking BCMA can effectively inhibit the growth of MM cells and reverse the immunosuppression microenvironment. In conclusion, BCMA is a suitable target for the treatment of multiple myeloma (MM).

### SUMMARY

The present invention provides a specific monoclonal antibody targeting BCMA. Specifically, the present invention relates to the following technical schemes:
1. An anti-BCMA antibody or an antigen-binding fragment thereof, comprising:
(i) an HCDR1, an HCDR2 and an HCDR3 comprised in a heavy chain variable region set forth in SEQ ID NO: 1; and/or an LCDR1, an LCDR2 and an LCDR3 comprised in a light chain variable region set forth in SEQ ID NO: 2;
(ii) an HCDR1, an HCDR2 and an HCDR3 comprised in a heavy chain variable region set forth in SEQ ID NO: 3; and/or an LCDR1, an LCDR2 and an LCDR3 comprised in a light chain variable region set forth in SEQ ID NO: 4;
(iii) an HCDR1, an HCDR2 and an HCDR3 comprised in a heavy chain variable region set forth in SEQ ID NO: 5; and/or an LCDR1, an LCDR2 and an LCDR3 comprised in a light chain variable region set forth in SEQ ID NO: 6; or
(iv) an HCDR1, an HCDR2 and an HCDR3 comprised in a heavy chain variable region; and/or an LCDR1, an LCDR2 and an LCDR3 comprised in a light chain variable region; wherein compared to the heavy chain variable region and/or the light chain variable region according to any of (i) to (iii), at least one CDR of the heavy chain variable region and/or the light chain variable region comprises a mutation that is a substitution, deletion or addition of one or more amino acids (e.g., a substitution, deletion or addition of 1, 2 or 3 amino acids) and is capable of retaining the binding affinity to BCMA; preferably, the mutation is a conservative amino acid mutation;
preferably, the CDRs are defined according to the IMGT, Kabat, Chothia or AbM numbering system;
preferably, the anti-BCMA antibody comprises the following CDRs according to the IMGT numbering system,
(1) an HCDR1 comprising or consisting of an amino acid sequence set forth in SEQ ID NO: 7 or a variant thereof,
an HCDR2 comprising or consisting of an amino acid sequence set forth in SEQ ID NO: 8 or a variant thereof,
an HCDR3 comprising or consisting of an amino acid sequence set forth in SEQ ID NO: 9 or a variant thereof,
an LCDR1 comprising or consisting of an amino acid sequence set forth in SEQ ID NO: 16 or a variant thereof,
an LCDR2 comprising or consisting of an amino acid sequence set forth in SEQ ID NO: 17 or a variant thereof, and
an LCDR3 comprising or consisting of an amino acid sequence set forth in SEQ ID NO: 18 or a variant thereof;
(2) an HCDR1 comprising or consisting of an amino acid sequence set forth in SEQ ID NO: 10 or a variant thereof,
an HCDR2 comprising or consisting of an amino acid sequence set forth in SEQ ID NO: 11 or a variant thereof,
an HCDR3 comprising or consisting of an amino acid sequence set forth in SEQ ID NO: 12 or a variant thereof,
an LCDR1 comprising or consisting of an amino acid sequence set forth in SEQ ID NO: 19 or a variant thereof,
an LCDR2 comprising or consisting of an amino acid sequence set forth in SEQ ID NO: 20 or a variant thereof, and
an LCDR3 comprising or consisting of an amino acid sequence set forth in SEQ ID NO: 21 or a variant thereof; or
(3) an HCDR1 comprising or consisting of an amino acid sequence set forth in SEQ ID NO: 13 or a variant thereof,
an HCDR2 comprising or consisting of an amino acid sequence set forth in SEQ ID NO: 14 or a variant thereof,
an HCDR3 comprising or consisting of an amino acid sequence set forth in SEQ ID NO: 15 or a variant thereof,
an LCDR1 comprising or consisting of an amino acid sequence set forth in SEQ ID NO: 22 or a variant thereof,
an LCDR2 comprising or consisting of an amino acid sequence set forth in SEQ ID NO: 17 or a variant thereof, and
an LCDR3 comprising or consisting of an amino acid sequence set forth in SEQ ID NO: 23 or a variant thereof,
wherein the variant is a sequence having at least 80%, 81%, 82%, 83%, 84%, 85%, 86%, 87%, 88%, 89% or 90%, preferably at least 91%, 92%, 93%, 94%, 95%, 96%, 97%, 98% or 99% sequence identity to the corresponding CDR sequence set forth in the SEQ ID NOs and retaining the binding affinity to BCMA, or an amino acid sequence having one or more (preferably 1, 2 or 3) conservative amino acid mutations (preferably substitutions, insertions or deletions) as compared to the corresponding CDR sequence set forth in the SEQ ID NOs and retaining the binding affinity to BCMA;
preferably, the heavy chain variable region and/or the light chain variable region of the antibody or the antigen-binding fragment thereof comprises FR regions from human, mouse or rabbit.
2. The anti-BCMA antibody or the antigen-binding fragment thereof according to item 1, wherein the heavy chain variable region of the antibody comprises or consists of a sequence set forth in SEQ ID NO: 1, SEQ ID NO: 3, SEQ ID NO: 5, SEQ ID NO: 24, SEQ ID NO: 26, SEQ ID NO: 28 or SEQ ID NO: 30, or a variant thereof; and
the light chain variable region of the antibody comprises or consists of a sequence set forth in SEQ ID NO: 2, SEQ ID NO: 4, SEQ ID NO: 6, SEQ ID NO: 25, SEQ ID NO: 27, SEQ ID NO: 29 or SEQ ID NO: 31, or a variant thereof;
wherein the variant is a sequence having at least 80%, 81%, 82%, 83%, 84%, 85%, 86%, 87%, 88%, 89% or 90%, preferably at least 91%, 92%, 93%, 94%, 95%, 96%, 97%, 98% or 99% sequence identity to the amino acid sequence of the corresponding antibody heavy chain or light chain variable region set forth in the SEQ ID NOs and retaining the binding affinity to BCMA, or an amino acid sequence having one or more (preferably 1, 2, 3, 4, 5, 6, 7, 8, 9 or 10) conservative amino acid mutations (preferably substitutions, insertions or deletions) as compared to the amino acid sequence of the corresponding antibody heavy chain or light chain variable region set forth in the SEQ ID NOs and retaining the binding affinity to BCMA.
3. The anti-BCMA antibody or the antigen-binding fragment thereof according to item 1 or 2, wherein
the heavy chain variable region of the antibody comprises an amino acid sequence set forth in SEQ ID NO: 1 or a variant thereof, and the light chain variable region of the antibody comprises an amino acid sequence set forth in SEQ ID NO: 2 or a variant thereof;
the heavy chain variable region of the antibody comprises an amino acid sequence set forth in SEQ ID NO: 3 or a variant thereof, and the light chain variable region of the antibody comprises an amino acid sequence set forth in SEQ ID NO: 4 or a variant thereof;
the heavy chain variable region of the antibody comprises an amino acid sequence set forth in SEQ ID NO: 5 or a variant thereof, and the light chain variable region of the antibody comprises an amino acid sequence set forth in SEQ ID NO: 6 or a variant thereof;
the heavy chain variable region of the antibody comprises an amino acid sequence set forth in SEQ ID NO: 24 or a variant thereof, and the light chain variable region of the antibody comprises an amino acid sequence set forth in SEQ ID NO: 25 or a variant thereof;
the heavy chain variable region of the antibody comprises an amino acid sequence set forth in SEQ ID NO: 26 or a variant thereof, and the light chain variable region of the antibody comprises an amino acid sequence set forth in SEQ ID NO: 27 or a variant thereof;
the heavy chain variable region of the antibody comprises an amino acid sequence set forth in SEQ ID NO: 28 or a variant thereof, and the light chain variable region of the antibody comprises an amino acid sequence set forth in SEQ ID NO: 29 or a variant thereof; or the heavy chain variable region of the antibody comprises an amino acid sequence set forth in SEQ ID NO: 30 or a variant thereof, and the light chain variable region of the antibody comprises an amino acid sequence set forth in SEQ ID NO: 31 or a variant thereof
4. The anti-BCMA antibody or the antigen-binding fragment thereof according to item 1 or 2, wherein the antibody comprises a heavy chain constant region and a light chain constant region that are derived from human IgG1, IgG2, IgG3 or IgG4; preferably, the heavy chain constant region is a human IgG1 heavy chain constant region, GenBank ACCESSION: AK303185.1, and the light chain constant region is an Ig1 kappa chain constant region, GenBank ACCESSION: MG815648.1.
5. The anti-BCMA antibody or the antigen-binding fragment thereof according to item 1 or 2, wherein the antibody is a monoclonal antibody, a partially or fully humanized antibody, a chimeric antibody, a single-chain antibody and a multispecific antibody (e.g., a bispecific antibody).
6. The anti-BCMA antibody or the antigen-binding fragment thereof according to item 1 or 2, wherein the antigen-binding fragment is selected from Fab, Fab′, F(ab′)₂, F(ab)₂, Fd, Fv, dAb, Fab/c, complementarity determining region fragment, scFv, scFv multimer, disulfide-stabilized Fv (dsFv), (dsFv)₂, bispecific dsFv (dsFv-dsFv′), diabody, disulfide-stabilized diabody (ds-diabody), multispecific antibody formed from a portion of an antibody comprising one or more CDRs, single-domain antibody (sdab), nanobody, domain antibody or bivalent domain antibody.
7. An isolated polypeptide or a variant thereof, selected from the group consisting of:
(1) an isolated polypeptide or a variant thereof, comprising sequences set forth in SEQ ID NO: 7, SEQ ID NO: 8 and SEQ ID NO: 9, wherein the polypeptide specifically binds to BCMA as part of an anti-BCMA antibody, the antibody further comprising sequences set forth in SEQ ID NO: 16, SEQ ID NO: 17 and SEQ ID NO: 18;
(2) an isolated polypeptide or a variant thereof, comprising sequences set forth in SEQ ID NO: 16, SEQ ID NO: 17 and SEQ ID NO: 18, wherein the polypeptide specifically binds to BCMA as part of an anti-BCMA antibody, the antibody further comprising sequences set forth in SEQ ID NO: 7, SEQ ID NO: 8 and SEQ ID NO: 9;
(3) an isolated polypeptide or a variant thereof, comprising sequences set forth in SEQ ID NO: 10, SEQ ID NO: 11 and SEQ ID NO: 12, wherein the polypeptide specifically binds to BCMA as part of an anti-BCMA antibody, the antibody further comprising sequences set forth in SEQ ID NO: 19, SEQ ID NO: 20 and SEQ ID NO: 21;
(4) an isolated polypeptide or a variant thereof, comprising sequences set forth in SEQ ID NO: 19, SEQ ID NO: 20 and SEQ ID NO: 21, wherein the polypeptide specifically binds to BCMA as part of an anti-BCMA antibody, the antibody further comprising sequences set forth in SEQ ID NO: 10, SEQ ID NO: 11 and SEQ ID NO: 12;
(5) an isolated polypeptide or a variant thereof, comprising sequences set forth in SEQ ID NO: 13, SEQ ID NO: 14 and SEQ ID NO: 15, wherein the polypeptide specifically binds to BCMA as part of an anti-BCMA antibody, the antibody further comprising sequences set forth in SEQ ID NO: 22, SEQ ID NO: 17 and SEQ ID NO: 23;
(6) an isolated polypeptide or a variant thereof, comprising sequences set forth in SEQ ID NO: 22, SEQ ID NO: 17 and SEQ ID NO: 23, wherein the polypeptide specifically binds to BCMA as part of an anti-BCMA antibody, the antibody further comprising sequences set forth in SEQ ID NO: 13, SEQ ID NO: 14 and SEQ ID NO: 15;
(7) an isolated polypeptide or a variant thereof, comprising a sequence set forth in SEQ ID NO: 1, wherein the polypeptide specifically binds to BCMA as part of an anti-BCMA antibody, the antibody further comprising a sequence set forth in SEQ ID NO: 2;
(8) an isolated polypeptide or a variant thereof, comprising a sequence set forth in SEQ ID NO: 2, wherein the polypeptide specifically binds to BCMA as part of an anti-BCMA antibody, the antibody further comprising a sequence set forth in SEQ ID NO: 1;
(9) an isolated polypeptide or a variant thereof, comprising a sequence set forth in SEQ ID NO: 3, wherein the polypeptide specifically binds to BCMA as part of an anti-BCMA antibody, the antibody further comprising a sequence set forth in SEQ ID NO: 4;
(10) an isolated polypeptide or a variant thereof, comprising a sequence set forth in SEQ ID NO: 4, wherein the polypeptide specifically binds to BCMA as part of an anti-BCMA antibody, the antibody further comprising a sequence set forth in SEQ ID NO: 3;
(11) an isolated polypeptide or a variant thereof, comprising a sequence set forth in SEQ ID NO: 5, wherein the polypeptide specifically binds to BCMA as part of an anti-BCMA antibody, the antibody further comprising a sequence set forth in SEQ ID NO: 6;
(12) an isolated polypeptide or a variant thereof, comprising a sequence set forth in SEQ ID NO: 6, wherein the polypeptide specifically binds to BCMA as part of an anti-BCMA antibody, the antibody further comprising a sequence set forth in SEQ ID NO: 5;
(13) an isolated polypeptide or a variant thereof, comprising a sequence set forth in SEQ ID NO: 24, wherein the polypeptide specifically binds to BCMA as part of an anti-BCMA antibody, the antibody further comprising a sequence set forth in SEQ ID NO: 25;
(14) an isolated polypeptide or a variant thereof, comprising a sequence set forth in SEQ ID NO: 25, wherein the polypeptide specifically binds to BCMA as part of an anti-BCMA antibody, the antibody further comprising a sequence set forth in SEQ ID NO: 24;
(15) an isolated polypeptide or a variant thereof, comprising a sequence set forth in SEQ ID NO: 26, wherein the polypeptide specifically binds to BCMA as part of an anti-BCMA antibody, the antibody further comprising a sequence set forth in SEQ ID NO: 27;
(16) an isolated polypeptide or a variant thereof, comprising a sequence set forth in SEQ ID NO: 27, wherein the polypeptide specifically binds to BCMA as part of an anti-BCMA antibody, the antibody further comprising a sequence set forth in SEQ ID NO: 26;
(17) an isolated polypeptide or a variant thereof, comprising a sequence set forth in SEQ ID NO: 28, wherein the polypeptide specifically binds to BCMA as part of an anti-BCMA antibody, the antibody further comprising a sequence set forth in SEQ ID NO: 29;
(18) an isolated polypeptide or a variant thereof, comprising a sequence set forth in SEQ ID NO: 29, wherein the polypeptide specifically binds to BCMA as part of an anti-BCMA antibody, the antibody further comprising a sequence set forth in SEQ ID NO: 28;
(19) an isolated polypeptide or a variant thereof, comprising a sequence set forth in SEQ ID NO: 30, wherein the polypeptide specifically binds to BCMA as part of an anti-BCMA antibody, the antibody further comprising a sequence set forth in SEQ ID NO: 31; or
(20) an isolated polypeptide or a variant thereof, comprising a sequence set forth in SEQ ID NO: 31, wherein the polypeptide specifically binds to BCMA as part of an anti-BCMA antibody, the antibody further comprising a sequence set forth in SEQ ID NO: 30,
wherein the variant is a sequence having at least 80%, 81%, 82%, 83%, 84%, 85%, 86%, 87%, 88%, 89% or 90%, preferably at least 91%, 92%, 93%, 94%, 95%, 96%, 97%, 98% or 99% sequence identity to the corresponding sequence set forth in the SEQ ID NOs and retaining the binding affinity to BCMA, or an amino acid sequence having one or more (preferably 1, 2, 3, 4, 5, 6, 7, 8, 9 or 10) conservative amino acid mutations (preferably substitutions, insertions or deletions) as compared to the corresponding sequence set forth in the SEQ ID NOs and retaining the binding affinity to BCMA.
8. A nucleic acid molecule encoding the antibody or the antigen-binding fragment thereof according to any of items 1 to 6, or the isolated polypeptide or the variant thereof according to item 7.
9. A vector comprising the nucleic acid molecule according to item 8.
10. A host cell comprising the nucleic acid molecule according to item 8 or the vector according to item 9.
11. A conjugate comprising the antibody or the antigen-binding fragment thereof according to any of items 1 to 6 and a conjugated moiety, wherein the conjugated moiety is a purification tag (e.g., His tag), a detectable label, a drug, a toxin, a cytokine, an enzyme or a combination thereof; preferably, the conjugated moiety is a radioisotope, a fluorescent substance, a chemiluminescent substance, a colored substance, a chemotherapeutic agent, a biotoxin, polyethylene glycol or an enzyme.
12. A fusion protein or a multispecific antibody (preferably a bispecific antibody) comprising the antibody or the antigen-binding fragment thereof according to any of items 1 to 6, wherein preferably, the fusion protein is a CAR construct that specifically binds to BCMA.
13. A kit comprising the antibody or the antigen-binding fragment thereof according to any of items 1 to 6, the conjugate according to item 11, or the fusion protein or the multispecific antibody according to item 12, wherein preferably, the kit further comprises a second antibody that specifically recognizes the antibody; optionally, the second antibody further comprises a detectable label, such as a radioisotope, a fluorescent substance, a chemiluminescent substance, a colored substance or an enzyme; preferably, the kit is configured for detecting the presence or level of BCMA in a sample.
14. A pharmaceutical composition comprising the antibody or the antigen-binding fragment thereof according to any of items 1 to 6, the conjugate according to item 11, or the fusion protein or the multispecific antibody according to item 12, wherein optionally, the pharmaceutical composition further comprises a pharmaceutically acceptable carrier and/or excipient; preferably, the pharmaceutical composition is in a form suitable for administration through subcutaneous injection, intradermal injection, intravenous injection, intramuscular injection or intralesional injection.
15. Use of the antibody or the antigen-binding fragment thereof according to any of items 1 to 6, the conjugate according to item 11, or the fusion protein or the multi specific antibody according to item 12 in treating and/or preventing a tumor (e.g., multiple myeloma), in preparing a medicament for treating and/or preventing a tumor (e.g., multiple myeloma), or in preparing a medicament for diagnosing a tumor.
16. A kit comprising (1) the antibody or the antigen-binding fragment thereof according to any of items 1 to 6, the conjugate according to item 11, or the fusion protein or the multispecific antibody according to item 12, and (2) an antibody or an antigen-binding fragment thereof against another antigen (e.g., CD38 and/or SLAMF7), and/or a cytotoxic agent, and optionally, a package insert.
17. A method for treating or preventing a tumor (e.g., multiple myeloma) comprising administering to a subject a therapeutically effective amount of the antibody or the antigen-binding fragment thereof according to any of items 1 to 6, the conjugate according to item 11, or the fusion protein or the multispecific antibody according to item 12.

It is to be understood that within the scope of the present invention, the above technical features of the present invention and the technical features specifically described hereinafter (as in the examples) may be combined with each other to constitute a new or preferred technical scheme. Due to the limited space, such schemes are not described herein.

The terms referred to in the present invention have the conventional meanings understood by those skilled in the art. Where a term has two or more definitions as used and/or acceptable in the art, the definitions of the terms used herein are intended to include all meanings.

It will be understood by those of ordinary skills in the art that the CDR regions of an antibody are responsible for the binding specificity of the antibody for an antigen. For a given heavy or light chain variable region sequence of an antibody, there are several methods for determining the CDR regions of the antibody, including the Kabat, IMGT, Chothia and AbM numbering systems. However, the application of all the definitions of CDRs for an antibody or variants thereof shall fall within the scope of the terms defined and used herein. If the amino acid sequence of the variable region of the antibody is given, those skilled in the art can generally determine which residues are contained in a particular CDR, without relying on any experimental data beyond the sequence itself.

As used herein, “antibody” or “antigen-binding fragment” refers to a polypeptide or polypeptide complex that specifically recognizes and binds to an antigen. The term “antibody” is used in a broad sense and includes immunoglobulins or antibody molecules including monoclonal or polyclonal human, humanized, complex and chimeric antibodies, and antibody fragments. The antibody may be an intact antibody or any antibody fragment, antigen-binding fragment or single chain thereof.

Thus, the term “antibody” includes any protein or peptide comprising a specific molecule that comprises at least a portion of an immunoglobulin molecule having biological activity for binding to an antigen. Such examples include, but are not limited to, complementarity determining regions (CDRs) of a heavy or light chain or a ligand binding portion thereof, heavy or light chain variable regions, heavy or light chain constant regions, framework regions (FRs) or any portion thereof, or at least a portion of a binding protein. In the present invention, the antibody includes murine, chimeric, humanized or fully human antibodies prepared by using techniques well known to those skilled in the art. Recombinant antibodies, such as chimeric and humanized monoclonal antibodies, including human and non-human portions, can be prepared by using recombinant DNA technology well known to those skilled in the art. The immunoglobulin molecules or antibody molecules of the present application may be of any type (e.g., IgG, IgE, IgM, IgD, IgA and IgY), or any class (e.g., IgG1, IgG2, IgG3, IgG4, IgA1 and IgA2) or subclass of immunoglobulin molecules.

The term “antibody fragment” or “antigen-binding fragment” includes, but is not limited to, F(ab′)₂, F(ab)₂, Fab′, Fab, Fv, Fd, dAb, Fab/c, complementarity determining region (CDR) fragment, single-chain Fv (scFv), disulfide-stabilized Fv fragment (dsFv), (dsFv)₂, bispecific dsFv (dsFv-dsFv′), diabody, disulfide-stabilized diabody (ds-diabody), scFv multimer (e.g., scFv dimer and scFv trimer), multi specific antibody formed from a portion of an antibody comprising one or more CDRs, nanobody, single-domain antibody (sdab), domain antibody, bivalent domain antibody, or any other antibody fragment that binds to an antigen but does not comprise an intact antibody structure. Regardless of the structure, an antigen-binding fragment includes any polypeptide or polypeptide complex capable of binding to the same antigen to which the parent antibody or the parent antibody fragment binds. The structure of dsFv is introduced by Mao C. S., et al., “Disulfide stabilized Fv Fragments (dsFv): a New Type of Engineering Antibody Fragments”. Progress in Biochemistry and Biophysics, 1998, 25(6):525-526. Antigen-binding fragments known as “domain antibodies” or dAbs, which contain only the VH or VL domains of the antibodies and are therefore smaller than, for example, Fab and scFv, are described by Holt, et al., “Domain antibodies: proteins for therapy”, Trends in Biotechnology (2003): vol. 21, No. 11: 484-490. dAbs are the smallest in known antigen-binding fragments of antibodies, with a molecular weight from 11 kDa to 15 kDa. The term “antibody fragment” includes aptamers, spiegelmers and diabodies. The term “antibody fragment” also includes any synthetic or genetically engineered protein that, like antibodies, binds to a specific antigen to form a complex. Typically, the antibody fragment has at least about 50 contiguous amino acids, preferably at least about 50 contiguous amino acids, more preferably at least about 80 contiguous amino acids, and most preferably at least about 100 contiguous amino acids, of the antibody of the present invention.

Embodiments of the present application provide a plurality of anti-BCMA antibodies comprising at least one antigen-binding domain targeting the BCMA antigen. An antigen-binding domain binding to the BCMA antigen may be an Fab, or an ScFv, or non-covalent pairing (Fv) between a heavy chain variable region (VH) and a light chain variable region (VL). Any of the above antibodies or polypeptides may also comprise additional polypeptides, e.g., a signal peptide at the N terminus of the antibody, which is used to direct secretion, or other heterologous polypeptides as described herein. In addition to intact antibodies, the present invention further includes immunocompetent antibody fragments or fusion proteins formed by the antibodies and other sequences. The present invention further provides other proteins or fusion expression products having the antibody of the present invention. Specifically, the present invention includes any protein or protein conjugate and fusion expression product (i.e., immunoconjugate and fusion expression product) having heavy and light chains with variable regions, so long as the variable regions are identical or have at least 90% homology, preferably at least 95% homology, to the variable regions of the heavy and light chains of the antibody of the present invention. Thus, the present invention includes molecules having monoclonal antibody light and heavy chain variable regions with CDRs, so long as the CDRs have 90% or more (preferably 95% or more, most preferably 98% or more) homology to the CDRs of the present invention.

The present invention further includes fragments, variants, derivatives and analogs of the antibodies. The antibody, the antigen-binding fragment, or the variant or derivative thereof of the present application includes, but is not limited to, polyclonal antibody, monoclonal antibody, multispecific antibody (e.g., bispecific antibody and trispecific antibody), human antibody, antibody of animal source, humanized antibody, primatized antibody, or chimeric antibody, CDR-grafted and/or modified antibody, single-chain antibody (e.g., scFv), diabody, epitope-binding fragments such as Fab, Fab′ and F(ab′)₂, Fd, Fv, single-chain Fv (scFv), single-chain antibody, disulfide-stabilized Fv (dsFv), fragments comprising VL or VH domains, fragments produced from Fab expression libraries, and anti-idiotypic (anti-Id) antibody. The antibody fragment, the antigen-binding fragment, the derivative or the analog of the present invention may be (i) a polypeptide in which one or more conservative or non-conservative amino acid residues (preferably conservative amino acid residues) are substituted, and such substituted amino acid residues may or may not be encoded by the genetic codes, or (ii) a polypeptide having a substituted group in one or more amino acid residues, or (iii) a polypeptide formed by fusing a mature polypeptide with another compound (such as a compound that prolongs the half-life of the polypeptide, e.g., polyethylene glycol), or (iv) a polypeptide formed by fusing an additional amino acid sequence with the polypeptide sequence (such as a leader sequence, a secretion sequence, a sequence for purifying the polypeptide, a proteinogenic sequence or a fusion protein with 6His tag). Such fragments, derivatives and analogs are well known to those skilled in the art according to the teachings of the present invention.

The antibody of the present invention refers to a polypeptide having binding activity to human BCMA and comprising the above CDR regions. The term further includes variants of the polypeptides having the same function as the antibody of the present invention and comprising the above CDR regions. These variations include (but are not limited to): deletion, insertion and/or substitution of one or more (generally 1-50, preferably 1-30, more preferably 1-20, and most preferably 1-10) amino acids, and addition of one or more (generally within 20, preferably within 10, and more preferably within 5) amino acids to the C terminus and/or N terminus. For example, in the art, substitutions with amino acids that are similar in properties generally do not alter the function of the protein. For another example, the addition of one or more amino acids to the C terminus and/or N terminus generally does not alter the function of the protein. The term further includes active fragments and active derivatives of the antibody of the present invention. Variants of the polypeptide include: homologous sequences, conservative variants, allelic variants, natural mutants, induced mutants, proteins encoded by DNA capable of hybridizing to DNA encoding the antibody of the present invention under high- or low-stringency conditions, and polypeptides or proteins obtained by using antisera against the antibody of the present invention.

For the purpose of comparing two or more amino acid sequences, the percentage “sequence homology” (also referred to herein as “amino acid homology”) between a first amino acid sequence and a second amino acid sequence can be calculated by dividing [the number of amino acid residues in a first amino acid sequence that are identical to the amino acid residues at the corresponding positions in a second amino acid sequence] by [the total number of amino acid residues in the first amino acid sequence] and multiplying by [100%], wherein each deletion, insertion, substitution or addition of an amino acid residue in the second amino acid sequence, as compared to the first amino acid sequence, is considered a difference in a single amino acid residue (position), i.e., an “amino acid difference” as defined in the present invention. Alternatively, the degree of sequence identity between two amino acid sequences can be calculated by using known computer algorithms, such as NCBI Multiple Alignment (https://www.ncbinlmnih.gov/tools/cobalt/cobalt.cgi?LINK_LOC=BlastHomeLink). Some other techniques, computer algorithms and arrangements for determining the degree of sequence identity are described, for example, in WO 04/037999, EP 0 967 284, EP 1 085 089, WO 00/55318, WO 00/78972, WO 98/49185 and GB 2 357 768-A. Typically, for the purpose of determining the percentage of “sequence identity” between two amino acid sequences according to the calculation methods set forth above, the amino acid sequence with the greatest number of amino acid residues is considered the “first” amino acid sequence, and the other amino acid sequence is considered the “second” amino acid sequence.

Furthermore, when determining the degree of sequence identity between two amino acid sequences, those skilled in the art may consider so-called “conservative” amino acid substitutions, which may generally be described as amino acid substitutions in which an amino acid residue is substituted with another amino acid residue having a similar chemical structure with little or no effect on the function, activity or other biological properties of the polypeptide. Such conservative amino acid substitutions are well known in the art, for example, from WO 04/037999, GB-A-3357 768, WO 98/49185, WO 00/46383 and WO 01/09300; and (preferred) types and/or combinations of such substitutions may be selected based on the relevant teachings of WO 04/037999 and WO 98/49185 and other references cited therein.

A “conservative amino acid substitution” is one in which an amino acid residue is substituted with an amino acid residue having a similar side chain. Families of amino acid residues having similar side chains have been defined in the art, including basic side chains (e.g., lysine, arginine and histidine), acidic side chains (e.g., aspartic acid and glutamic acid), uncharged polar side chains (e.g., glycine, asparagine, glutamine, serine, threonine, tyrosine and cysteine), non-polar side chains (e.g., alanine, valine, leucine, isoleucine, proline, phenylalanine, methionine and tryptophan), branched β side chains (e.g., threonine, valine and isoleucine) and aromatic side chains (e.g., tyrosine, phenylalanine, tryptophan and histidine). Thus, non-essential amino acid residues of an immunoglobulin polypeptide are preferably substituted with other amino acid residues from the same side chain family. In other embodiments, a string of amino acids may be substituted with a structurally similar string of amino acids that differ in sequence and/or composition of the side chain family.

Non-limiting examples of conservative amino acid substitutions are provided in the following table, where a similarity score of 0 or higher indicates that there is a conservative substitution between the two amino acids.

| | C | G | P | S | A | T | D | E | N | Q | H | K | R | V | M | I | L | F | Y | W |
|---|---|---|---|---|---|---|---|---|---|---|---|---|---|---|---|---|---|---|---|---|
| W | −8 | −7 | −6 | −2 | −6 | −5 | −7 | −7 | −4 | −5 | −3 | −3 | 2 | −6 | −4 | −5 | −2 | 0 | 0 | 17 |
| Y | 0 | −5 | −5 | −3 | −3 | −3 | −4 | −4 | −2 | −4 | 0 | −4 | −5 | −2 | −2 | −1 | −1 | 7 | 10 | |
| F | −4 | −5 | −5 | −3 | −4 | −3 | −6 | −5 | −4 | −5 | −2 | −5 | −4 | −1 | 0 | 1 | 2 | 9 | | |
| L | −6 | −4 | −3 | −3 | −2 | −2 | −4 | −3 | −3 | −2 | −2 | −3 | −3 | 2 | 4 | 2 | 6 | | | |
| I | −2 | −3 | −2 | −1 | −1 | 0 | −2 | −2 | −2 | −2 | −2 | −2 | −2 | 4 | 2 | 5 | | | | |
| M | −5 | −3 | −2 | −2 | −1 | −1 | −3 | −2 | 0 | −1 | −2 | 0 | 0 | 2 | 6 | | | | | |
| V | −2 | −1 | −1 | −1 | 0 | 0 | −2 | −2 | −2 | −2 | −2 | −2 | −2 | 4 | | | | | | |
| R | −4 | −3 | 0 | 0 | −2 | −1 | −1 | −1 | 0 | 1 | 2 | 3 | 6 | | | | | | | |
| K | −5 | −2 | −1 | 0 | −1 | 0 | 0 | 0 | 1 | 1 | 0 | 5 | | | | | | | | |
| H | −3 | −2 | 0 | −1 | −1 | −1 | 1 | 1 | 2 | 3 | 6 | | | | | | | | | |
| Q | −5 | −1 | 0 | −1 | 0 | −1 | 2 | 2 | 1 | 4 | | | | | | | | | | |
| N | −4 | 0 | −1 | 1 | 0 | 0 | 2 | 1 | 2 | | | | | | | | | | | |
| E | −5 | 0 | −1 | 0 | 0 | 0 | 3 | 4 | | | | | | | | | | | | |
| D | −5 | 1 | −1 | 0 | 0 | 0 | 4 | | | | | | | | | | | | | |
| T | −2 | 0 | 0 | 1 | 1 | 3 | | | | | | | | | | | | | | |
| A | −2 | 1 | 1 | 1 | 2 | | | | | | | | | | | | | | | |
| S | 0 | 1 | 1 | 1 | | | | | | | | | | | | | | | | |
| P | −3 | −1 | 6 | | | | | | | | | | | | | | | | | |
| G | −3 | 5 | | | | | | | | | | | | | | | | | | |
| C | 12 | | | | | | | | | | | | | | | | | | | |

In some embodiments, the conservative substitution is preferably a substitution in which one amino acid within the following groups (a)-(e) is substituted with another amino acid residue within the same group: (a) small aliphatic, non-polar or weakly polar residues: Ala, Ser, Thr, Pro and Gly, (b) polar, negatively charged residues and (uncharged) amides thereof: Asp, Asn, Glu and Gln, (c) polar, positively charged residues: His, Arg and Lys, (d) bulky aliphatic, nonpolar residues: Met, Leu, Ile, Val and Cys, and (e) aromatic residues: Phe, Tyr and Trp.

Particularly preferred conservative substitutions are as follows: Ala is substituted with Gly or Ser; Arg is substituted with Lys; Asn is substituted with Gln or His; Asp is substituted with Glu; Cys is substituted with Ser; Gln is substituted with Asn; Glu is substituted with Asp; Gly is substituted with Ala or Pro; His is substituted with Asn or Gln; Ile is substituted with Leu or Val; Leu is substituted with Ile or Val; Lys is substituted with Arg, Gln or Glu; Met is substituted with Leu, Tyr or Ile; Phe is substituted with Met, Leu or Tyr; Ser is substituted with Thr; Thr is substituted with Ser; Trp is substituted with Tyr; Tyr is substituted with Trp; and/or Phe is substituted with Val, Ile or Leu.

In some embodiments, the antibody of the present invention may be conjugated with a therapeutic agent, a prodrug, a peptide, a protein, an enzyme, a virus, a lipid, a biological response modifier, a pharmaceutical agent or PEG. The antibody of the present invention can be linked or fused to a therapeutic agent, and the therapeutic agent may comprise a detectable label such as a radioactive label, an immunomodulator, a hormone, an enzyme, an oligonucleotide, a photoactive therapeutic or diagnostic agent, a cytotoxic agent that can be a drug or a toxin, an ultrasound enhancing agent, a nonradioactive label, a combination thereof and other such ingredients known in the art.

In some embodiments, the anti-BCMA antibody of the present invention, for example, has one or more of the following advantages:
(a) the antibody of the present invention has excellent biological activity and specificity, high affinity for BCMA, higher binding activity than existing BCMA antibodies (such as 83A10 antibody), and no significant potential toxic and side effects.
(b) the antibody of the present invention is humanized, exhibiting retained high affinity and reduced immunogenicity.
(c) the antibody of the present invention has binding activity to BCMA of cynomolgus monkeys, and is thus helpful to the test and the quality control test in animal models.
(d) the antibody of the present invention has good stability, particularly in acidic and heating conditions.

### BRIEF DESCRIPTION OF THE DRAWINGS

FIG. 1 shows expression plasmids of full-length BCMAs of human (A), monkey (B) and mouse (C) corresponding to the construction of stable transfected cell lines HEK293 huBCMA, HEK293 cynoBCMA and HEK293 mBCMA.

FIG. 2 shows the identification of BCMA expression of human, monkey and mouse BCMAs in stable transfected cell lines HEK293 huBCMA, HEK293 cynoBCM and HEK293 mBCMA by FACS.

FIG. 3 shows the human-monkey cross-reactivity assay of the antibodies in the hybridoma supernatants of 18F6G3H9, 202E11H10E3 and 211B11H10G5 by FACS.

FIG. 4 shows the competitive ELISA assay for chimeric antibodies c-mAb1, c-mAb2 and c-mAb3 inhibiting the binding of the natural ligand APRIL to human BCMA.

FIG. 5 shows the competitive ELISA assay for chimeric antibodies c-mAb1, c-mAb2 and c-mAb3 inhibiting the binding of the positive control antibody 83A10 to human BCMA.

FIG. 6 shows the competitive ELISA assay for chimeric antibody c-mAb3 inhibiting the binding of the positive control antibody 83A10 to monkey BCMA.

FIG. 7 shows the competitive ELISA assay for humanized antibodies hu-mAb 1, hu-mAb2, hu-mAb3 and hu-mAb4 inhibiting the binding of the natural ligand APRIL to human BCMA.

FIG. 8 shows the competitive ELISA assay for humanized antibodies hu-mAb 1, hu-mAb2, hu-mAb3 and hu-mAb4 inhibiting the binding of the positive control antibody 83A10 to human BCMA.

FIG. 9 shows the competitive ELISA assay for humanized antibodies hu-mAb3 and hu-mAb4 inhibiting the binding of the positive control antibody 83A10 to monkey BCMA.

FIG. 10 shows the ADCC effect of effector cells NK92MI-CD16a on target cells NCI-H929 mediated by humanized monoclonal antibodies hu-mAb1, hu-mAb2, hu-mAb3 and hu-mAb4.

FIG. 11 shows the ADCC effect of effector cells NK92MI-CD16a on target cells U266B1 mediated by humanized monoclonal antibodies hu-mAb1, hu-mAb2, hu-mAb3 and hu-mAb4.

### DETAILED DESCRIPTION

The present invention will be described in detail below by way of examples. It will be understood by those of ordinary skills in the art that the following examples are for illustrative purposes only. The spirit and scope of the present invention are defined by the claims. Unless otherwise stated, the methods used in the following examples are conventional methods and the reagents used are commercially available reagents.

### Example 1: Preparation of Materials

### 1.1 BCMA ECD Molecules

Recombinant human BCMA-huFc and BCMA-mFc fusion proteins corresponding to amino acids 1-54 of human BCMA (SEQ ID NO: 32) are hereinafter referred to as huBCMA-huFc and huBCMA-mFc, respectively; recombinant cynomolgus monkey BCMA-huFc and BCMA-mFc corresponding to amino acids 1-53 of cynomolgus monkey BCMA (SEQ ID NO: 33) are hereinafter referred to as cynoBCMA-huFc and cynoBCMA-mFc, respectively; recombinant mouse BCMA-huFc and BCMA-mFc fusion proteins corresponding to amino acids 1-49 of mouse BCMA (SEQ ID NO:34) are hereinafter referred to as mBCMA-huFc and mBCMA-mFc, respectively. The above sequence information is from the U.S. National Center for Biotechnology Information. Recombinant human hu BCMA-his fusion proteins (ACRO Biosystem, Cat. No. BCA-H522y) of human BCMA and recombinant cynomolgus monkey cynoBCMA-his (ACRO Biosystem, Cat. No. BCA-052H7) of monkey BCMA were applied to characterization and analysis. These materials were used for binding and affinity measurements. The amino acid sequence information of the BCMA ECD molecules is shown in Table 1.

TABLE 1
BCMA ECD sequence information

| Species | Sequence |
|---|---|
| Human | MLQMAGQCSQNEYFDSLLHACIPCQLRCSSNTPPLTCQRYCNASVINSVKGTNA (SEQ |
| | ID NO: 32) |
| Cynomolgus | MLQMARQCSQNEYFDSLLHDCKPCQLRCSSTPPLTCQRYCNASMTNSVKGMN (SEQ ID |
| monkey | NO: 33) |
| Mouse | MAQQCFHSEYFDSLLHACKPCHLRCSNPPATCQPYCDPSVTSSVKGTYT (SEQ ID |
| | NO: 34) |
| hu-Fc | IEGRMDPKSCDKTHTCPPCPAPELLGGPSVFLFPPKPKDTLMISRTPEVTCVVVDVSHED |
| | PEVKFNWYVDGVEVHNAKTKPREEQYNSTYRVVSVLTVLHQDWLNGKEYKCKVSNK |
| | ALPAPIEKTISKAKGQPREPQVYTLPPSRDELTKNQVSLTCLVKGFYPSDIAVEWESNGQP |
| | ENNYKTTPPVLDSDGSFFLYSKLTVDKSRWQQGNVFSCSVMHEALHNHYTQKSLSLSP |
| | GK (SEQ ID NO: 35) |
| mFc | EPRGPTIKPCPPCKCPAPNLLGGPSVFIFPPKIKDVLMISLSPIVTCVVVDVSEDDPDVQIS |
| | WFVNNVEVHTAQTQTHREDYNSTLRVVSALPIQHQDWMSGKEFKCKVNNKDLPAPIE |
| | RTISKPKGSVRAPQVYVLPPPEEEMTKKQVTLTCMVTDFMPEDIYVEWTNNGKTELNY |
| | KNTEPVLDSDGSYFMYSKLRVEKKNWVERNSYSCSVVHEGLHNHHTTKSFSRTPGK |
| | (SEQ ID NO: 36) |

### 1.2 Construction of BCMA Cell Lines

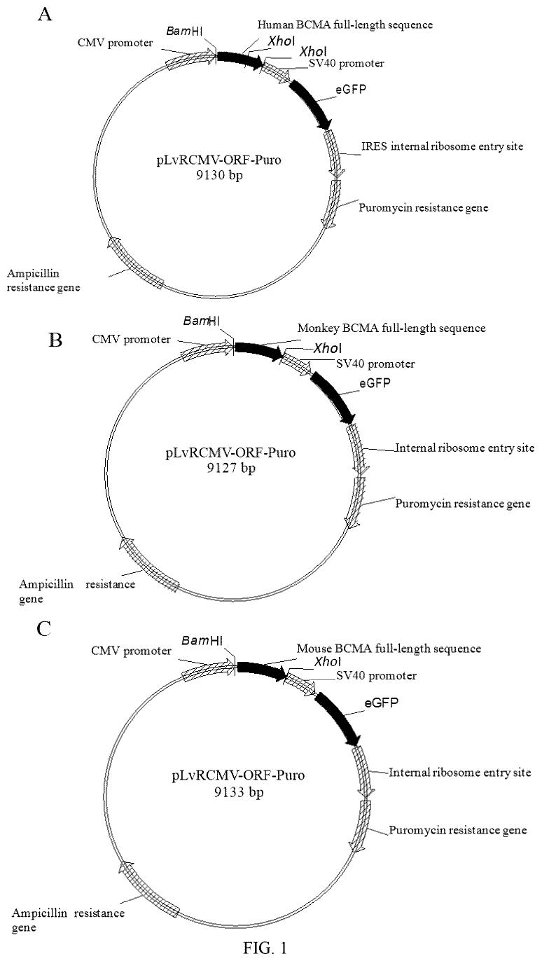

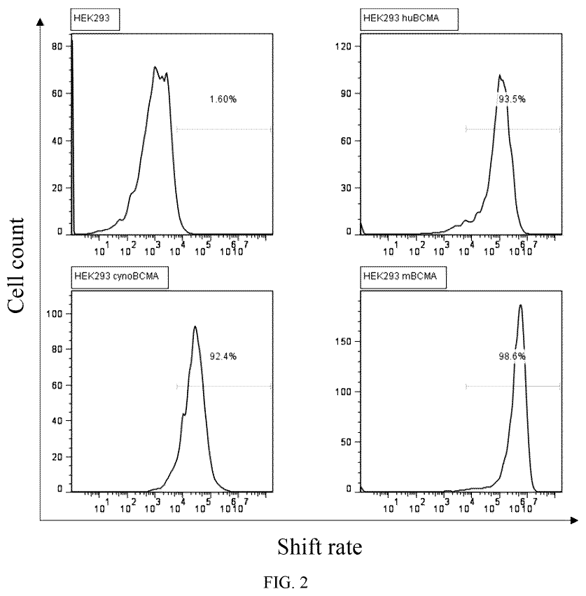

Vectors presenting human BCMA (FIG. 1A), cynomolgus monkey BCMA (FIG. 1B) and mouse BCMA (FIG. 1C) were transiently transfected into HEK293 cells using standard methods. Green fluorescent protein-positive monoclones were selected by FACS and cultured. Then the cell surface receptor expression was detected using an anti-human BCMA antibody (Biolegend, Cat. No. 357504), anti-cynomolgus monkey BCMA antibody 83A10 (see 83A10 derived from U.S. Pat. No. WO2018083204A1 for the sequence, VH corresponding to SEQ ID NO: 19, VL corresponding to SEQ ID NO: 29, GenBank No. AK303185.1 for the human IgG1 heavy chain constant region and GenBank No. MG815648.1 for the human κ light chain constant region) and anti-mouse BCMA antibody (R&D System, Cat. No. MAB593). HEK293 cell lines stably expressing the protein was constructed (FIG. 2), hereinafter referred to as HEK293 huBCMA, HEK293 cynoBCMA and HEK293 mBCMA.

As seen from FIG. 2, relative to the negative control cell HEK293, the shift rates of the stable transfected cell lines HEK293 huBCMA, HEK293 cynoBCMA and HEK293 mBCMA were 93.5%, 92.4% and 98.6%, respectively, indicating that the HEK293 huBCMA, HEK293 cynoBCMA and HEK293 mBCMA cell lines stably express BCMA on the cell surface.

### Example 2: Preparation and Separation of Hybridoma Expressing BCMA Monoclonal Antibody

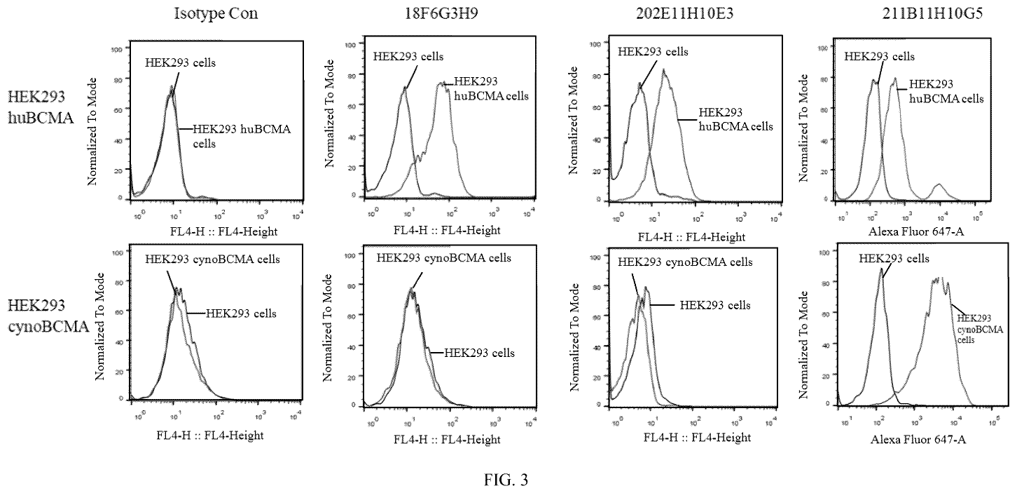

The immunization was performed as follows: The antigen huBCMA-his was emulsified with an adjuvant and then used to immunize BALB/c, C57BL/6, SJL and ICR mice and SD rats (purchased from Model Organisms) subcutaneously/intraperitoneally at multiple sites. The serum titer in the immunized mice/rats was monitored, and animals failing the titer test were subjected to further immunizations. After the titer requirement was met, spleen cells of the animals were collected and electrically fused with myeloma (Sp2/0) cells. Polyclonal hybridoma cells were obtained by HAT screening. Positive polyclones were selected through ELISA and FACS and subcloned by limiting dilution to give stable single positive hybridoma cells. Positive clones were selected by ELISA and FACS. After the screening, three monoclonal cell lines 18F6G3H9, 202E11H10E3 and 211B11H10G5 having high BCMA-binding activity were obtained, as shown in Table 2 and FIG. 3. Hybridoma supernatants from these three monoclonal cell lines were subjected to sequencing, cloning and expression to further construct a human IgG1 chimeric antibody and to complete the monoclonal antibody humanization. The sequencing results are shown in Example 3.

### Example 3: Sequencing of Anti-BCMA Antibody V-Gene Sequence Clones

The RNA was extracted by the Trizol method, and the cDNA was obtained by reverse transcription. The cDNA was amplified to give the heavy and light chain variable regions. A library was constructed using the PCR product and quality control procedures were conducted. High throughput sequencing was conducted on a Miseq 2×300 PE system. By bioinformatics analysis, the sequencing results were aligned to the IMGT database to determine the CDR sequences. The V region sequencing results of the 3 mouse BCMA antibodies are as follows, with the CDR sequences shown in Table 3:

(1) Clone 18F6G3H9:
BCMA-HC1
SEQ ID NO: 1
EVQLQQSGPELVKPGTSVEMSCKASGYTFTDYNIHWVRQRHGESLEWLGYINPNNGVT
NFNQKFKGKATLTVNKSSSTAYMELRSLTSDDSAVYYCSSGLFDGYPFPYWGQGTLVTV
SA; wherein, the CDR1, CDR2 and CDR3 are underlined (SEQ ID NOs: 7, 8 and 9).
BCMA-LC1
SEQ ID NO: 2
DIVLTQSPASLVVSLGQRATISCRASKSVSTSGYNYIHWYQQKPGQPPKLLIFLASNLKSGV
PARFSGGGSGTDFTLNIHPVEEDDAATYFCQHNRELPYTFGGGTKVEIK;
wherein, the
CDR1, CDR2 and CDR3 are underlined (SEQ ID NOs: 16, 17 and 18).
(2) Clone 202E11H10E3:
BCMA-HC2
SEQ ID NO: 3
QVQLQQSDAELVKPGTSVKISCKVSGYTFTDHTIHWMKQRPEQGLEWIGYIYPRNGNTK
YNEKFKGKATLTADKSSSTAYMHLNSLTSEDSAVYFCARYDYDGYFDVWGTGTTVTVSS;
wherein, the CDR1, CDR2 and CDR3 are underlined (SEQ ID NOs: 10, 11 and 12).
BCMA-LC2
SEQ ID NO: 4
DVVMTQTPLSLPVSLGDQASISCRSSQSHVHSDGHTYLHWYLQKPGQSPELLIYKVSNRF
SGVPDRFSGSGSGTDFTLKISRVEAEDLGVYFCSQNTHFPWTFGGGTKLEIK; wherein, the
CDR1, CDR2 and CDR3 are underlined (SEQ ID NOs: 19, 20 and 21).
(3) Clone 211B11H10G5:
BCMA-HC3
SEQ ID NO: 5
EVQLQQSGPELVKPGASVKILCKASGYTITDYNMDWVKRSHGKSLEWIGNINPHNGGSI
YNQKFKGKATLTVDKSSSTAFMELRSLTSEDTAVYYCARGDATLVLDYWGQGTSLTVSS;
wherein, the CDR1, CDR2 and CDR3 are underlined (SEQ ID NOs: 13, 14 and 15).
BCMA-LC3
SEQ ID NO: 6
DIVLTQSPASLAVSLGQRATISCRASKSVSTSGLSYMHWYQQKPGQPPKVLIYLASNLESG
VPARFSGSGSGTDFTLNIHPVEEEDAATYYCQHSRELPPTFGAGTKLELK; wherein, the
CDR1, CDR2 and CDR3 are underlined (SEQ ID NOs: 22, 17 and 23).

TABLE 2
ELISA results for hybridoma supernatants

| | Dilution factor of hybridoma cell supernatant | | | | | | | | | |
|---|---|---|---|---|---|---|---|---|---|---|
| | Stock | | | | | | | | Blank | Immobilized |
| Clone | solution | 1:32 | 1:9 | 1:27 | 1:81 | 1:243 | 1:729 | 1:2187 | Medium | antigen |
| 18F6G3H9 | 2.51 | 2.41 | 2.22 | 2.00 | 1.53 | 1.04 | 0.48 | 0.31 | 0.06 | huBCMA-huFc |
| | 0.08 | 0.05 | 0.07 | 0.06 | 0.08 | 0.06 | 0.07 | 0.08 | 0.05 | cynoBCMA-huFc |
| 202E11H10E3 | 2.86 | 2.84 | 2.80 | 2.78 | 2.56 | 2.13 | 1.53 | 0.79 | 0.07 | huBCMA-huFc |
| | 0.75 | 0.31 | 0.18 | 0.15 | 0.14 | 0.15 | 0.13 | 0.12 | 0.07 | cynoBCMA-huFc |
| 211B11H10G5 | 2.66 | 2.63 | 2.15 | 1.83 | 1.67 | 1.06 | 0.49 | 0.26 | 0.06 | huBCMA-huFc |
| | 2.97 | 2.92 | 2.75 | 2.51 | 1.85 | 1.00 | 0.45 | 0.19 | 0.05 | cynoBCMA-huFc |

TABLE 3
CDR sequences of mouse anti-BCMA antibodies (according to IMGT numbering system)

| | | | SEQ ID | | | SEQ ID | | | SEQ ID |
|---|---|---|---|---|---|---|---|---|---|
| Clone | CDR1 | | NO .: | CDR2 | | NO.: | CDR3 | | NO.: |
| 18F6G3H9 | VH | GYTFTDYN | 7 | VH | INPNNGVT | 8 | VH | CSSGLFDGYPFPYW | 9 |
| | VL | KSVSTSGYNY | 16 | VL | LAS | 17 | VL | CQHNRELPYTF | 18 |
| 202E11H10E3 | VH | GYTFTDHT | 10 | VH | IYPRNGNT | 11 | VH | CARYDYDGYFDVW | 12 |
| | VL | QSHVHSDGHTY | 19 | VL | KVS | 20 | VL | CSQNTHFPWTF | 21 |
| 211B11H10G5 | VH | GYTITDYN | 13 | VH | INPHNGGS | 14 | VH | CARGDATLVLDYW | 15 |
| | VL | KSVSTSGLSY | 22 | VL | LAS | 17 | VL | CQHSRELPPTF | 23 |

### Example 4: Characterization of Mouse BCMA Antibody-Expressing Cells that Bind to BCMA

The ascites of the mice were purified by protein A affinity chromatography to give the BCMA antibodies. The concentrations of the purified mice BCMA antibodies were determined by UV absorbance at 280 nm and the corresponding extinction coefficient for each protein. The purities of the antibodies were assessed by SDS-PAGE (purity>90% for all) and the endotoxin content in the antibody preparations was determined by LAL assay (endotoxin content<3 EU/mg for all). Mouse monoclonal antibodies 18F6G3H9, 202E11H10E3 and 211B11H10G5 were obtained by purifying the hybridoma supernatants and the 3 mouse BCMA antibodies were assessed for their binding to BCMA antigens, modified BCMA-expressing cell line HEK293 cynoBCMA and cancer cell lines U266B1 and NCI-H929, by using ELISA, BiaCore molecular binding assay and flow cytometry, wherein U266B1 and NCI-H929 were both purchased from the China Center for Type Culture Collection. The purpose of the screening assay was to identify, at the molecular and cellular level, the cross-reactivities of the mouse antibodies with human BCMA and with cynomolgus monkey BCMA.

### 4.1 Determination of Mouse Monoclonal Antibodies Using Indirect ELISA

The affinities and species cross-reactivities of the 3 mouse antibodies for BCMA antigens were assessed by ELISA.

Materials: BCMA ECD: huBCMA-huFc and cynoBCMA-huFc; PBS buffer (pH 7.4; Gibco, C10010500BT); BSA (Bovogen, BSA0.1), TWEEN 20 (SINOPHARM, 30189328) and HRP goat anti-mouse IgG antibody (Abclonal, AS003), TMB (BD, 55214), H2504 (SINOPHARM, 10021618).

Procedures: Human, mouse and monkey BCMA antigens were prepared into 0.5 μg/mL coating solutions with PBS buffer. The coating solutions were added to a microplate at 100 μL/well and incubated at 4° C. overnight. The residual coating solution was discarded before 3% BSA was added at 300 μL/well, and the plate was blocked for 3 h at room temperature. The microplate was washed once with PBST (PBS containing 0.1% of TWEEN 20) at 300 μL/well. The three mouse BCMA antibodies were serially 3-fold diluted from 10 μg/mL to the 7th concentration. The dilutions were added to the microplate at 100 μL/well. After a 1-h incubation at room temperature, the microplate was washed thrice with PBST at 300 μL/well. The HRP goat anti-mouse IgG antibody was 25,000-fold diluted with 1% BSA-PBST and added at 100 μL/well. After another 1-h incubation at room temperature, the microplate was washed thrice with PBST at 300 μL/well and dried. The TMB chromogenic solution was added at 100 μL/well. After a 5-min reaction at room temperature, 2 M H₂SO₄ was added at 100 μL/well to terminate the reaction. The microplate was placed on a microplate reader (Molecular Devices, SPECTRA Max plus 384) and the absorbance OD450 value was read at 450 nm wavelength. The results are shown in Table 4.

TABLE 4
Binding activity comparison of mouse
antibodies to different BCMA species

| | huBCMA-huFc, | cynoBCMA-huFc, | mBCMA-huFc, |
|---|---|---|---|
| Sample | EC50 | EC50 | EC50 |
| 18F6G3H9 | 70.5 pM | — | — |
| 202E11H10E3 | 76.4 pM | — | — |
| 211B11H10G5 | 49.2 pM | 30.89 pM | — |

The results show that 18F6G3H9, 202E11H10E3 and 211B11H10G5 mouse antibodies can bind to human BCMA with high binding activity, and the EC₅₀ values were all lower than 80 pM. 211B11H10G5 can also bind to monkey BCMA.

### 4.2 Determination of Mouse Monoclonal Antibodies Using FACS

The affinities of the 3 mouse antibodies for cell surface BCMA antigen were assessed using FACS. Materials: cell lines NCI-H929, U266B1, HEK293 cynoBCMA and HEK293; PBS buffer; FBS (fetal bovine serum; Gibco, 10099141); PE goat anti-mouse IgG Fc (Biolegend, 405307). Procedures: The cells were resuspended in PBS to prepare cell suspensions containing 200,000 cells. Mouse BCMA antibodies were added to a 96-well plate at 50 μL/well and serially 3-fold diluted from 3000 nM. The plate was incubated for 1 h at 4° C. 150 μL of precooled PBS containing 1% of FBS was added into the 96-well plate after the co-incubation with the primary antibody was finished. The mixtures were centrifuged at 300×g for 5 min and the supernatants were discarded. The above procedures were repeated once. The fluorescent secondary antibody PE goat anti-mouse IgG Fc was added at 80 μL/well. The plate was incubated at 4° C. for 30 min. 150 μL of precooled PBS containing 1% of FBS was added into the 96-well plate after the co-incubation with the secondary antibody was finished. The mixtures were uniformly mixed with a pipette. The 96-well plate was centrifuged at 300×g for 5 min in a plate centrifuge and the supernatants were discarded. The above procedures were repeated once. The cells were resuspended in 100 μL of PBS containing 1% of FBS and detected on a flow cytometry (BD Accuri™ C6). The results are shown in Table 5.

TABLE 5
Binding activity comparison of mouse antibodies
to human or monkey BCMA-expressing cell lines

| | U266B1, | NCI-H929, | HEK293 cynoBCMA, |
|---|---|---|---|
| Sample | EC50 | EC50 | EC50 |
| 18F6G3H9 | 1.6 nM | 3.1 nM | — |
| 202E11H10E3 | 1 nM | 1.1 nM | — |
| 211B11H10G5 | 24.5 nM | 11.9 nM | 30.89 nM |

The results show that the mouse antibodies 18F6G3H9, 202E11H10E3 and 211B11H10G5 can bind to NCI-H929 and U266B1 cells with high or medium-low human BCMA expression with affinities of 50 nM or less. 211B11H10G5 can bind to monkey BCMA. The affinity is comparable to that of the corresponding antibody for human BCMA.

### Example 5: Expression and Purification of BCMA Chimeric Antibodies

### 5.1 Preparation of Chimeric Antibody Plasmids

Chimeric heavy and light chains were constructed by ligating the cDNAs of VH and VL regions obtained by PCR against hybridomas mouse antibodies 18F6G3H9, 202E11H10E3 and 211B11H10G5 with the encoding DNAs of the human IgG1 heavy chain constant region (GenBank No. AK303185.1) and the kappa light chain constant region (GenBank No. MG815648.1), respectively, to obtain the expression plasmids of the heavy and light chains of the corresponding human-mouse chimeric antibodies c-mAb 1, c-mAb2 and c-mAb3. The vector pcDNA3.1(-) (purchased from Invitrogen), or other eukaryotic expression vector may also be used with the corresponding mouse light and heavy chain constant regions being replaced with the constant region sequences of human IgG1 heavy chain or kappa light chain.

### 5.2 Expression and Purification of Chimeric Antibodies

Plasmid extraction was performed using EndoFree Plasmid Giga Kit (Qiagen, Cat. No. 12391) following the manufacturer's manual. CHO-S cells were cultured in CD CHO medium (Gibco, Cat. No. 10743-029) in a 37° C., 5% CO2 incubator according to the manufacturer's manual. After the cells were prepared, CHO-S cells were co-transfected with plasmids containing heavy/light chain sequences to express anti-BCMA monoclonal chimeric antibodies c-mAb1, c-mAb2 and c-mAb3. The day after transfection, the culture temperature was adjusted to 32° C. and 3.5% 2×EFC+(Gibco, Cat. No. A2503105) was added daily. After 14 days of culture, expression supernatants were harvested by centrifugation at 800×g. The supernatants were filtered through a 0.22 μm filter membrane, and purified by protein A affinity chromatography and cation exchange chromatography to give the BCMA antibodies in the culture supernatants. The concentrations of the purified chimeric antibodies were determined by UV absorbance at 280 nm and the corresponding extinction coefficient for each protein. The purity and homogeneity of the antibodies were assessed by SDS-PAGE and SE-HPLC. The antibodies were further purified by a second purification through ion exchange and SEC with Superdex 200 to prepare antibody samples of higher purities for later use.

### Example 6: Binding Activity of BCMA Chimeric Antibodies to BCMA

The binding activities of human-mouse chimeric monoclonal antibodies to BCMA antigens, the modified monkey BCMA-expressing cell line HEK293 cynoBCMA and the hematologic tumor cell lines U266B1 and NCI-H929, were assessed using ELISA, BiaCore and flow cytometry. The purpose of the screening assay was to identify, at the molecular and cellular level, the cross-reactivities of the antibodies with human BCMA and with cynomolgus monkey BCMA.

### 6.1 Indirect ELISA—Determination of Chimeric Monoclonal Antibodies

The affinities of the 3 chimeric antibodies were assessed using ELISA to select antibodies with proper affinity and cross-species reactivity.

Materials: BCMA ECD: huBCMA-mFc and cynoBCMA-mFc; PBS buffer; BSA (Bovogen, Cat. No. BSA0.1), TWEEN 20 (SINOPHARM, Cat. No. 30189328) and mouse anti-human IgG Fc antibody [HRP] mAb (Genscript, Cat. No. A01854), TMB (BD, Cat. No. 55214), H2504 (SINOPHARM, Cat. No. 10021618).

Procedures: Human, mouse and monkey BCMA antigens were prepared into 0.5 μg/mL coating solutions with PBS buffer. The coating solutions were added to a microplate at 100 μL/well and incubated at 4° C. overnight. The residual coating solution was discarded before 3% BSA was added at 300 μL/well, and the plate was blocked for 3 h at room temperature. The microplate was washed once with PB ST (PBS containing 0.1% of TWEEN 20) at 300 μL/well. The human-mouse chimeric monoclonal antibodies were serially 3-fold diluted from 10 μg/mL to the 9th concentration. The dilutions were added to the microplate at 100 μL/well. After a 1-h incubation at room temperature, the microplate was washed thrice with PB ST at 300 μL/well. The mouse anti-human IgG Fc antibody [HRP] mAb was 25,000-fold diluted with 1% BSA-PBST and added at 100 μL/well. After another 1-h incubation at room temperature, the microplate was washed thrice with PBST at 300 μL/well and dried. The TMB chromogenic solution was added at 100 μL/well. After a 5-min reaction at room temperature, 2 M H₂SO₄ was added at 100 μL/well to terminate the reaction. The microplate was placed on a microplate reader (Molecular Devices, SPECTRA Max plus 384) and the absorbance OD450 value was read at 450 nm wavelength. The results are shown in Table 6.

TABLE 6
Binding activity comparison of chimeric
antibodies to different BCMA species

| | Corresponding clones | huBCMA-mFc, | cynoBCMA-mFc, |
|---|---|---|---|
| Sample | of chimeric antibodies | EC50 | EC50 |
| c-mAb1 | 18F6G3H9 | 647.4 pM | — |
| c-mAb2 | 202E11H10E3 | 427.4 pM | — |
| c-mAb3 | 211B11H10G5 | 793.7 pM | 722.5 pM |

The results demonstrate that the chimeric antibodies c-mAb1, c-mAb2 and c-mAb3 are all capable of binding to huBCMA-mFc with EC50 values less than 1000 pM. c-mAb3 can bind to cynoBCMA-mFc with a calculated EC50 value of 722.5 pM.

### 6.2 FACS—Screening of Chimeric Monoclonal Antibodies

The affinities of the 3 chimeric antibodies for cell surface BCMA antigen were assessed using FACS.

Materials: cell lines NCI-H929, U266B1, HEK293 cynoBCMA and HEK293; PBS buffer; FBS (fetal bovine serum; Gibco, 10099141); PE anti-human IgG Fc (Biolegend, 409304).

Procedures: The cells were resuspended in PBS, and the cell density was adjusted to 2×10 5 cells/well. Human-mouse chimeric monoclonal antibodies were added to a 96-well plate at 50 μL/well and serially 3-fold diluted from 3000 nM to the 9th concentration. The plate was incubated for 1 h at 4° C. 150 μL of precooled PBS containing 1% of FBS was added into the 96-well plate after the co-incubation with the primary antibody was finished. The mixtures were centrifuged at 300×g for 5 min and the supernatants were discarded. The above procedures were repeated once. The fluorescent secondary antibody PE anti-human IgG Fc (Biolegend, 409304) was added at 80 μL/well. The plate was incubated at 4° C. for 30 min. 150 μL of precooled PBS containing 1% of FBS was added into the 96-well plate after the co-incubation with the secondary antibody was finished. The 96-well plate was centrifuged at 300×g for 5 min and the supernatants were discarded. The above procedures were repeated once. The cells were resuspended in 100 μL of PBS containing 1% of FBS and detected on a flow cytometry (BD Accuri™ C6). The results are shown in Table 7.

TABLE 7
Binding activity comparison of chimeric antibodies
to human or monkey BCMA-expressing cell lines

| | Corresponding | | | HEK293 |
|---|---|---|---|---|
| | clones of chimeric | NCI-H929, | U266B1, | cynoBCMA, |
| Sample | antibodies | EC50 | EC50 | EC50 |
| c-mAb1 | 18F6G3H9 | 7.5 nM | 1.9 nM | — |
| c-mAb2 | 202E11H10E3 | 5.7 nM | 14.7 nM | — |
| c-mAb3 | 211B11H10G5 | 8.25 nM | 81.5 nM | 2.66 nM |

The results demonstrate that the chimeric antibodies can bind to cells with either high human BCMA expression (NCI-H929) or medium-low BCMA expression (U266B1). c-mAb3 can also bind to monkey BCMA-expressing cell line HEK293 cynoBCMA with a calculated EC50 value of 2.66 nM.

### Example 7: BCMA Chimeric Antibodies Inhibiting Binding of APRIL Ligand to BCMA

The abilities of anti-BCMA chimeric antibodies to block the binding of April to BCMA antigen were assessed by APRIL competitive binding ELISA.

Materials: Biotin labeling kit (DoJindo, LK03); soluble human April (ACRO, Cat. No. APL-H5244); cynoBCMA-his (ACRO, BCA-052H7) and huBCMA-his (ACRO, BCA-H522y); PBS buffer; BSA (Bovogen, BSA0.1); TWEEN 20 (SINOPHARM, 30189328); Streptavidin-Peroxidase Polymer (Sigma-Aldrich, 52438-250UG); TMB Substrate Reagent Set (RUO) (BD, 555214); H2504 (SINOPHARM, 10021618).

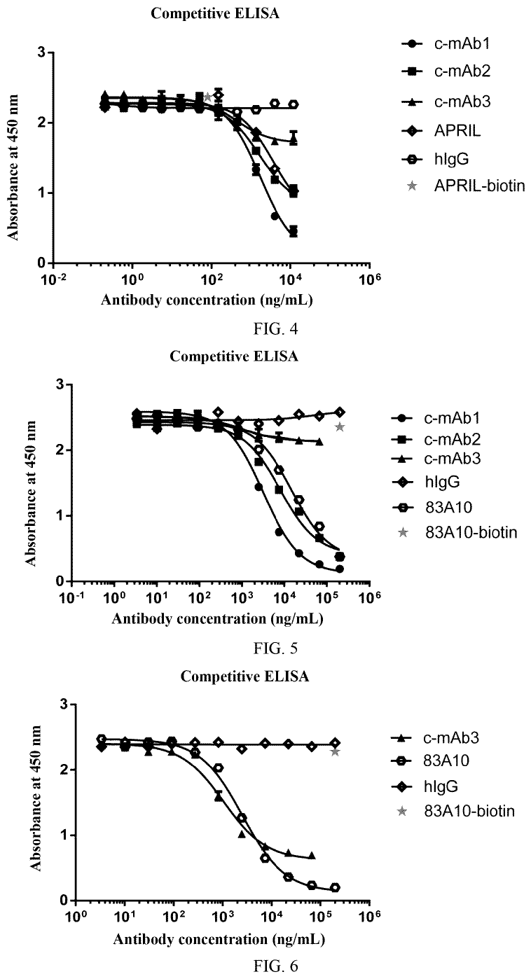

Procedures: The soluble human APRIL protein was labeled with biotin according to the manual to give APRIL-biotin. The 96-well microplate was treated with 100 μL of 0.5 μg/mL huBCMA-his in PBS and incubated overnight at 4° C. The plate was then washed thrice with PBS washing buffer containing 0.1% of Tween-20 and blocked with 300 μL of PBS containing 1% of BSA for 2 h. 100 μL of each BCMA antibody was added to the plate. After a 1-h incubation at 37° C., APRIL-biotin was added at 80 ng/well and the plate was incubated for 1 h at 37° C. Unbound APRIL-biotin was washed with PBS washing buffer containing 0.1% of Tween-20 before the secondary antibody streptavidin-peroxidase polymer (Sigma-Aldrich, S2438-250UG) was added. The plate was incubated at 37° C. for 1 h, and unbound secondary antibody was washed with PBS washing buffer containing 0.1% of Tween-20. The TMB chromogenic solution was added at 100 μL/well. After a 5-min reaction at room temperature, 2 M H₂SO₄ was added at 100 μL/well to terminate the reaction. The microplate was placed on a microplate reader (Molecular Devices, SPECTRA Max plus 384) and the absorbance OD450 value was read at 450 nm wavelength. The results are shown in Table 8 and FIG. 4.

TABLE 8
Comparison of activity of chimeric antibodies to competitively
inhibiting binding of APRIL ligand to human BCMA

| | Corresponding clones of | |
|---|---|---|
| Sample | chimeric antibodies | IC50 (ng/mL) |
| APRIL | — | 24.67 |
| c-mAb1 | 18F6G3H9 | 11.37 |
| c-mAb2 | 202E11H10E3 | 10.77 |
| c-mAb3 | 211B11H10G5 | 2.748 |

IgG from human serum (hIgG; Sigma 14506) purified by protein A affinity chromatography was used as the negative control in this study. The results show that the chimeric antibodies c-mAb1 and c-mAb2 can inhibit the binding of APRIL to huBCMA-his, and when the antibody concentration reached 12 μg, c-mAb 1 exhibited an 80% competition, and c-mAb2 exhibited a 60% competition. c-mAb3 showed affinity for huBCMA-his comparable to those of c-mAb1 and c-mAb2 but failed to effectively inhibit the binding of APRIL to huBCMA-his, suggesting that the antibody c-mAb3 may have a binding site different from APRIL to huBCMA-his.

### Example 8: Human-Mouse Chimeric Monoclonal Antibodies Inhibiting Binding of Positive Antibody 83A10 to BCMA

The abilities of anti-BCMA antibodies to block the binding of positive control antibody 83A10 (Patent No. WO2014122144A1) to BCMA antigen were assessed.

Materials: Biotin labeling kit (DoJindo, LK03); positive antibody 83A 10 (Patent No. WO2014122144A1); cynoBCMA-his (ACRO, BCA-052H7) and huBCMA-his (ACRO, BCA-H522y); PBS buffer; BSA (Bovogen, BSA0.1); TWEEN 20 (SINOPHARM, 30189328); Streptavidin—Peroxidase Polymer (Sigma-Aldrich, 52438-250UG); TMB Substrate Reagent Set (RUO) (BD, 555214); H2504 (SINOPHARM, 10021618).

Procedures: The soluble human 83A10 protein (for the sequence, see the antibody of clone No. 83A10 in U.S. Pat. No. WO2018083204A1 with VH corresponding to SEQ ID NO:19, VL corresponding to SEQ ID NO:29, the heavy chain constant region being human IgG1 heavy chain constant region and the light chain constant region being human kappa light chain constant region) was labeled with biotin according to the manual to give 83A10-biotin. The 96-well microplate was treated with 100 μL of 0.5 μg/mL huBCMA-his in PBS and incubated overnight at 4° C. The plate was then washed thrice with PBS washing buffer containing 0.1% of Tween-20 and blocked with 300 μL of PBS containing 1% of BSA for 2 h. 100 μL of each BCMA antibody was added to the plate. After a 1-h incubation at 37° C., 5 ng/mL 83A10-biotin was added to each well and the plate was incubated for 1 h at 37° C. Unbound 83A10-biotin was washed with PBS washing buffer containing 0.1% of Tween-20 before the secondary antibody streptavidin-peroxidase polymer was added. The plate was incubated at 37° C. for 1 h, and unbound secondary antibody was washed with PBS washing buffer containing 0.1% of Tween-20. The TMB chromogenic solution was added at 100 μL/well. After a 5-min reaction at room temperature, 2 M H₂SO₄ was added at 100 μL/well to terminate the reaction. The microplate was placed on a microplate reader (Molecular Devices, SPECTRA Max plus 384) and the absorbance OD450 value was read at 450 nm wavelength. The results are shown in Table 9, FIG. 5 and FIG. 6.

TABLE 9
Comparison of activity of chimeric antibodies to competitively
inhibiting binding of 83A10 to human BCMA

| | Corresponding clones | IC50 (ng/mL) | |
|---|---|---|---|
| Sample | of chimeric antibodies | huBCMA-his | cynoBCMA-his |
| 83A10 | — | 15011 | 2274 |
| c-mAb1 | 18F6G3H9 | 3163 | — |
| c-mAb2 | 202E11H10E3 | 7997 | — |
| c-mAb3 | 211B11H10G5 | 1003 | 1052 |

The results demonstrate that the antibodies c-mAb1 and c-mAb2 can inhibit the binding of 83A10 to huBCMA-his, and when the antibody concentration reached 7 μg/mL, c-mAb 1 exhibited a 70% competition and c-mAb2 exhibited a 40% competition. c-mAb3 showed affinity for huBCMA-his comparable to those of c-mAb1 and c-mAb2 but failed to effectively inhibit the binding of 83A10 to huBCMA-his, suggesting that the antibody c-mAb3 may have a binding site different from 83A10 to huBCMA-his. At 7 μg/mL, c-mAb3 could compete with 83A10 for 50% of binding to cynoBCMA-his.

### Example 9: Construction and Preparation of Humanized Monoclonal Antibodies

The purpose of humanization design was to transform the original mouse sequence into a human sequence to reduce the immunogenicity by 3D modeling and database alignment. This was implemented by changing the mouse CDR (complementarity determining region) sequences into humanized sequences through CDR grafting.

Heavy Chain Design:
the original heavy chain mVH sequence of molecule c-mAb 1 was modified to give 1 humanized sequence huVH1 (SEQ ID NO: 24, VH1);
the original heavy chain mVH sequence of molecule c-mAb2 was modified to give 1 humanized sequence huVH2 (SEQ ID NO: 26, VH2); and
the original heavy chain mVH sequence of molecule c-mAb3 was modified to give 2 humanized sequences huVH3 (SEQ ID NO: 28, VH3) and huVH4 (SEQ ID NO: 30, VH4).

Light Chain Design:
the original light chain mVL sequence of molecule c-mAb 1 was modified to give 1 humanized sequence huVL1 (SEQ ID NO: 25, VL1);
the original light chain mVL sequence of molecule c-mAb2 was modified to give 1 humanized sequence huVL2 (SEQ ID NO: 27, VL2); and
the original light chain mVL of molecule c-mAb3 was modified to give 2 humanized sequences huVL3 (SEQ ID NO: 29, VL3) and huVL4 (SEQ ID NO: 31, VL4).

The 8 sequences designed above were combined into 4 humanized antibodies hu-mAb1(VH1+VL1), hu-mAb2(VH2+VL2), hu-mAb3(VH3+VL3) and hu-mAb4(VH4+VL4) for subsequent expression validation. c-mAb1 is the parent antibody to hu-mAb1, c-mAb2 is the parent antibody to hu-mAb2, and c-mAb3 is the parent antibody to hu-mAb3 and hu-mAb4.

The specific sequences are shown below.

TABLE 10
Sequences of humanized monoclonal antibodies

| | | |
|---|---|---|
| hu-mAb1 | VH1 | DIVLTQSPDSLAVSLGREATINCRASKSVSTSGYNYIHWYQQKPGQP |
| | (SEQ ID | PKLLIFLASNLKSGVPDRFSGSGSGTDFTLTISSLQAEDVAVYYCQH |
| | NO: 24) | NRELPYTFGGGTKVEIK |
| | VL1 | EVQLVESGGALVKPGGSLRLSCKASGYTITDYNMDWVRQSPGKGL |
| | (SEQ ID | EWIGNINPHNGGSIYNQKFQGRATITVDKSTSTAYMELSSLRSEDTA |
| | NO: 25) | VYYCARGDATLVLDYWGQGTLVTVSS |
| hu-mAb2 | VH2 | QVQLVQSGAEVKKPGSSVKVSCKVSGYTFTDHTIHWMKQAPGQG |
| | (SEQ ID | LEWIGYIYPRNGNTKYNEKFKGKATLTADKSTSTAYMELSSLRSED |
| | NO: 26) | TAVYFCARYDYDGYFDVWGQGTLVTVSS |
| | VL2 | DVVMTQTPLSLSVTPGQPASISCRSSQSHVHSDGHTYLHWYLQKPG |
| | (SEQ ID | QSPELLIYKVSNRFSGVPDRFSGSGSGTDFTLKISRVEAEDVGVYFC |
| | NO: 27) | SQNTHFPWTFGGGTKVEIK |
| hu-mAb3 | VH3 | QVQLVQSGAEVKKPGASVKVSCKASGYTITDYNMDWVRQAPGQ |
| | (SEQ ID | GLEWIGNINPHNGGSIYNQKFQGRATLTVDKSTSTAYMELSSLRSED |
| | NO: 28) | TAVYYCARGDATLVLDYWGQGTTVTVSS |
| | VL3 | DIQLTQSPSSLSASVGDRATITCRASKSVSTSGLSYMHWYQQKPGK |
| | (SEQ ID | APKVLIYLASNLESGVPARFSGSGSGTDFTLTISSVQPEDFATYYCQH |
| | NO: 29) | SRELPPTFGQGTKLELK |
| hu-mAb4 | VH4 | EVQLVQSGAEVKKPGASVKVSCKASGYTFTDYNIHWVRQAPGQG |
| | (SEQ ID | LEWLGYINPNNGVTNFNQKFKGRATLTVDKSTSTAYMELRSLRSD |
| | NO: 30) | DTAVYYCSSGLFDGYPFPYWGQGTLVTVSS |
| | VL4 | EIVLTQSPATLSVSPGREATLSCRASKSVSTSGLSYMHWYQQKPGQ |
| | (SEQ ID | APRVLIYLASNLESGVPARFSGSGSGTEFTLTISSLQSEDAAVYYCQH |
| | NO: 31) | SRELPPTFGQGTKLEIK |

The humanized antibody expression plasmids were expressed in ExpiCHO-S(ATCC, NO. CCL-61) cells, and the cultures were purified to give the humanized antibodies. By ELISA, Biacore and cell affinity assays, 4 humanized antibodies were obtained (named “hu-mAb1, hu-mAb2, hu-mAb3 and hu-mAb4”).

The results show that the humanized antibodies have comparable or superior affinity and specificity to the mouse antibodies. See Example 10.

### Example 10: Assessment of Stability in Acidic Condition and Thermal Condition for Humanized Antibodies

Humanized antibodies were assessed for stability in acidic condition and thermal condition according to conventional methods. When the antibody molecules were subjected to protein A affinity chromatography, the eluted antibody solution was not neutralized in the acid elution step (using a citric acid buffer at pH 3.5). After 30 min, a sample was collected and 1/10 volume of 1 M Tris-HCl (pH 8.0) was added to the sample for neutralization. The sample was analyzed by HPLC-SEC. As shown in Table 11, the humanized antibody molecules hu-mAb1, hu-mAb2, hu-mAb3 and hu-mAb4 did not aggregate or degrade after 30 min of treatment at pH 3.5 with a purity of >95%, indicating that the antibodies are stable in acidic environments. Meanwhile, no aggregation or degradation was found after treatment at 40° C. for 14 days, and the purities were all >95%, indicating that the antibodies can remain stable in a 40° C. environment, as shown in Table 12.

TABLE 11
Stability assessment of humanized antibodies in acidic condition

| | pH 3.5, 30 min | |
|---|---|---|
| Sample | Pre-treatment purity by SEC | Post-treatment purity by SEC |
| hu-mAb1 | 99.99% | 95.42% |
| hu-mAb2 | 99.39% | 99.32% |
| hu-mAb3 | 95.67% | 95.59% |
| hu-mAb4 | 99.72% | 99.65% |

TABLE 12
Thermal stability assessment of humanized antibodies

Thermal stability test at 40° C.

| | Purity by SEC | Purity by SEC | Purity by SEC |
|---|---|---|---|
| Sample | on Day 0 | on Day 7 | on Day 14 |
| hu-mAb1 | 99.99% | 99.27% | 99.29% |
| hu-mAb2 | 99.39% | 99.08% | 99.08% |
| hu-mAb3 | 95.67% | 95.47% | 93.77% |
| hu-mAb4 | 98.84% | 96.95% | 96.58% |

### Example 11: Determination of Affinity of Humanized Antibody Molecules by ELISA

In this example, the affinities of the 4 humanized antibodies were assessed by ELISA.

Materials: BCMA ECD: huBCMA-his and cynoBCMA-his; PBS buffer; BSA (Bovogen, BSA0.1), TWEEN 20 (SINOPHARM, 30189328); HRP-conjugated 6*His, His-Tag antibody (Proteintech, HRP-66005-100), TMB (BD, 55214); H₂SO₄ (SINOPHARM, 10021618).

Procedures: huBCMA-his and cynoBCMA-his antigens were prepared into 0.5 μg/mL coating solutions with PBS (pH 7.4). The coating solutions were added to a microplate at 100 μL/well and incubated at 4° C. overnight. The residual coating solution was discarded before 3% BSA was added at 300 μL/well, and the plate was blocked for 3 h at room temperature. The microplate was washed once with PB ST at 300 The stock humanized monoclonal antibody solutions were serially 3-fold diluted to the 11th concentration. The dilutions were added to the microplate at 100 μL/well. After a 1-h incubation at room temperature, the microplate was washed thrice with PBST at 300 The HRP-conjugated 6*His, His-Tag antibody was 25,000-fold diluted with 1% BSA-PBST and added at 100 After another 1-h incubation at room temperature, the microplate was washed thrice with PB ST at 300 μL/well and dried. The TMB chromogenic solution was added at 100 μL. After a 5-min reaction at room temperature, 2 M H2504 was added at 100 μL/well to terminate the reaction. The microplate was placed on a microplate reader (Molecular Devices, SPECTRA Max plus 384) and the absorbance OD450 value was read at 450 nm wavelength. The results are shown in Table 13.

TABLE 13
Binding activity comparison of humanized
antibodies to human BCMA and monkey BCMA

| | huBCMA-his | cynoBCMA-his |
|---|---|---|
| Sample | (EC50, pM) | (EC50, pM) |
| hu-mAb1 | 17.65 | — |
| hu-mAb2 | 2.71 | — |
| hu-mAb3 | 22.63 | 30.27 |
| hu-mAb4 | 22.47 | 18.6 |
| c-mAb1 | 1.58 | — |
| c-mAb2 | 25.7 | — |
| c-mAb3 | 15.55 | 24.84 |
| 83A10 | 25.32 | 34.73 |
| hIgG | — | — |

The results demonstrate that through humanization, the humanized monoclonal antibodies exhibited binding activities to huBCMA-his and cynoBCMA-his comparable to those of the parent antibodies, with affinity differences within 3 folds of the change, and humanized monoclonal antibodies with improved affinity were surprisingly obtained. The affinity was improved by 10%-90% as compared with that of the positive control antibody 83A10. Meanwhile, the cross-species reactivity is retained, and the affinity for binding monkey antigen is significantly improved as compared with those of the human-mouse chimeric monoclonal antibodies.

### Example 12: Determination of Affinity of Humanized Monoclonal Antibodies by BiaCore

In this example, the antigen-antibody binding kinetics and affinity were determined by the BIACORE method.

Materials: human BCMA/TNFRSF17 protein (ACRO, BCA-H522y); cynomolgus/rhesus macaque BCMA/TNFRSF17 protein (ACRO, BCA-052H7); Sereis S Sensor Chip CM5 (GE, BR100530); anti-histidine antibody (GE, 28995056); HBS-EP(10×) (GE, BR-1006-69); glycine, 10 mM, pH 1.5 (GE, BR100354).

Procedures: The anti-histidine antibody (GE, His capture Kit, Cat. No. 28995056) was conjugated to the Sereis S Sensor Chip CMS to capture the test articles, thus acquiring the binding kinetic and affinity data of the antigen, as an analyte, to the test articles. The initial concentration for detecting the binding of the antigen and the test articles was 10 nM. On this basis, the antigens were serially 2-fold diluted to concentrations 10 nM, 5 nM, 2.5 nM, 1.25 nM and 0.625 nM. The antigens were loaded in an ascending order of concentration, and 1 negative control (1×HBS-EP+buffer) and 1 replicate concentration (generally the lowest concentration) were set. The Start up flushing process (1×HBS-EP+buffer) was performed at least thrice before loading to equilibrate the system. The association and dissociation tendencies between the antigen and the test articles were detected. When the dissociation was completed, a regeneration reagent was loaded for regeneration before the detection of the next concentration was performed. After the detection was finished, the data were fitted by using a 1:1 Binding fitting mode in data analysis software (Biacore T200 Evaluation Software). The results are shown in Table 14.

TABLE 14
Affinity of humanized antibodies
for human BCMA and monkey BCMA

| Sample | Antigen | ka(1/Ms) | kd(1/s) | KD(M) |
|---|---|---|---|---|
| 83A10 | huBCMA-his | 2.05E+06 | 2.15E−03 | 1.05E−09 |
| c-mAb1 | | 8.18E+06 | 2.02E−03 | 2.47E−10 |
| c-mAb2 | | 4.68E+07 | 6.79E−02 | 1.45E−09 |
| c-mAb3 | | 4.69E+06 | 1.78E−03 | 3.80E−10 |
| hu-mAb1 | | 9.49E+06 | 5.17E−04 | 5.44E−11 |
| hu-mAb2 | | 2.24E+07 | 2.58E−02 | 1.15E−09 |
| hu-mAb3 | | 5.90E+06 | 2.35E−03 | 3.99E−10 |
| hu-mAb4 | | 8.32E+06 | 1.72E−02 | 2.07E−09 |
| 83A10 | cynoBCMA- | 4.95E+06 | 4.94E−03 | 9.97E−09 |
| c-mAb3 | his | 3.54E+06 | 3.73E−04 | 1.06E−10 |
| hu-mAb1 | | — | — | — |
| hu-mAb2 | | — | — | — |
| hu-mAb3 | | 3.02E+06 | 7.73E−04 | 2.56E−10 |
| hu-mAb4 | | 2.75E+06 | 2.37E−03 | 8.61E−10 |

The affinity assay results for human/monkey BCMA showed that the affinities of the humanized anti-BCMA antibodies of the present invention were one to two orders of magnitude higher than the control antibody 83A10 (Patent No. WO2014122144A1), indicating stronger affinities.

### Example 13: Humanized Antibodies Inhibiting Binding of APRIL Ligand to BCMA

The abilities of humanized anti-BCMA antibodies to block the binding of APRIL to BCMA antigen were assessed by APRIL competitive binding ELISA.

Materials: Biotin labeling kit (DoJindo, LK03); soluble human APRIL (ACRO, Cat. No. APL-H5244); cynoBCMA-his (ACRO, BCA-052H7) and huBCMA-his (ACRO, BCA-H522y); TMB Substrate Reagent Set (BD, 555214).

Procedures: The procedures were the same as those in Example 7, with the human-mouse chimeric monoclonal antibodies being replaced with the humanized monoclonal antibodies.

TABLE 15
Comparison of activity of humanized antibodies to competitively
inhibiting binding of APRIL ligand to human BCMA

| Sample | IC50 (ng/mL) |
|---|---|
| APRIL | 3700 |
| hu-mAb1 | 1646 |
| hu-mAb2 | 2758 |
| hu-mAb3 | 918.4 |
| hu-mAb4 | 476.4 |

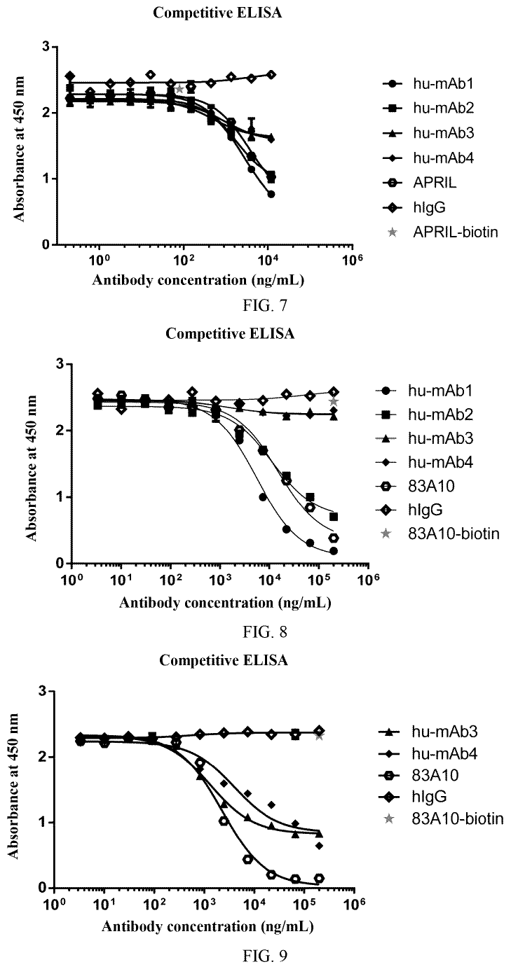

The results are shown in Table 15 and FIG. 7. The results demonstrate that the humanized antibodies hu-mAb 1, hu-mAb2, hu-mAb3 and hu-mAb4 can inhibit the binding of APRIL to huBCMA-his with consistent tendencies with that of the chimeric antibodies. When the antibody concentration reached 12 μg/mL, hu-mAb 1 and hu-mAb2 exhibited 70% and 60% competitions, respectively. hu-mAb3 and hu-mAb4 exhibited high affinities for huBCMA-his but failed to significantly inhibit the binding of APRIL to huBCMA-his, indicating that antibodies hu-mAb3 and hu-mAb4 may also have different binding sites from APRIL to huBCMA-his.

### Example 14: Humanized Antibodies Inhibiting Binding of Positive Antibody 83A10

The procedures were the same as those in Example 8, with the human-mouse chimeric monoclonal antibodies being replaced with the humanized monoclonal antibodies.

TABLE 16
Comparison of activity of humanized antibodies to competitively
inhibiting binding of 83A10 to human and monkey BCMAs

| | huBCMA-his, | cynoBCMA-his, |
|---|---|---|
| Sample | IC50 (ng/mL) | IC50 (ng/mL) |
| 83A10 | 9802 | 2265 |
| hu-mAb1 | 5776 | — |
| hu-mAb2 | 8346 | — |
| hu-mAb3 | 1374 | 1316 |
| hu-mAb4 | 2304 | 3963 |

The results are shown in Table 16, FIG. 8 and FIG. 9. The results demonstrate that the humanized antibodies can inhibit the binding of 83A10 to huBCMA-his with consistent tendencies with the chimeric antibodies. When the antibody concentration reached 7 μg/mL, hu-mAb 1 and hu-mAb2 exhibited 60% and 30% competitions. hu-mAb3 and hu-mAb4 exhibited high affinity for huBCMA-his but failed to significantly inhibit the binding of 83A10 to huBCMA-his, suggesting that antibodies hu-mAb3 and hu-mAb4 may have different binding sites from APRIL to huBCMA-his.

### Example 15: Assessment of Affinity of Humanized Antibodies for Cells

The affinities of the 4 humanized monoclonal antibodies for cell surface BCMA antigen were assessed using FACS.

Materials: cell lines NCI-H929, HEK293 cynoBCMA and HEK293; buffer: 1% FBS-PBS, pH 7.4; PE anti-human IgG Fc (Biolegend, 409304)

Procedures: Preparation of cell suspensions: The cells were resuspended in PBS, and the cell density was adjusted to 2×10⁵ cells/well. Mouse antibodies were added to a 96-well plate at 50 and serially 3-fold diluted from 3000 nM. The plate was incubated for 1 h at 4° C. 150 μL of precooled PBS containing 1% of FBS was added into the 96-well plate after the co-incubation with the primary antibody was finished. The mixtures were centrifuged at 300×g for 5 min and the supernatants were discarded. The above procedures were repeated once. The fluorescent secondary antibody PE anti-human IgG Fc (Biolegend, 409304) was added at 80 μL/well. The plate was incubated at 4° C. for 30 min. 150 μL of precooled PBS containing 1% of FBS was added into the 96-well plate after the co-incubation with the secondary antibody was finished. The mixtures were uniformly mixed with a pipette. The 96-well plate was centrifuged at 300×g for 5 min in a plate centrifuge and the supernatants were removed. The above procedures were repeated once. The cells were resuspended in 100 μL of PBS containing 1% of FBS and detected on a flow cytometry (BD Accuri™ C6). The results are shown in Table 17.

TABLE 17
Binding activity comparison of humanized antibodies
to human/monkey BCMA-expressing cell lines

| | NCI-H929 | HEK293 cynoBCMA |
|---|---|---|
| Sample | EC50 (nM) | EC50 (nM) |
| c-mAb1 | 7.5 | — |
| c-mAb2 | 5.7 | — |
| c-mAb3 | 8.25 | 2.66 |
| hu-mAb1 | 2.45 | — |
| hu-mAb2 | 0.83 | — |
| hu-mAb3 | 8.07 | 3.76 |
| hu-mAb4 | 8.12 | 6.86 |
| 83A10 | 8.69 | 8.42 |

The results indicate that the affinities of the humanized antibodies corresponding to c-mAb 1, c-mAb2 and c-mAb3 varied within 3 folds. The affinities of c-mAb1, c-mAb2 and c-mAb3 and the corresponding humanized monoclonal antibodies, hu-mAb1, hu-mAb2, hu-mAb3 and hu-mAb4 for NCI-H929 cell line were all superior to that of 83A10; the parent antibody c-mAb3 with human-monkey cross-reactivity and its humanized monoclonal antibodies hu-mAb3 and hu-mAb4, which also exhibited human-monkey cross-reactivities, also have affinities for HEK293 cynoBCMA superior to that of 83A 10.

### Example 16: ADCC Effect of Humanized Antibodies

Effects of the antibodies for mediating ADCC before and after humanization were assessed.

Materials: Cell lines NCI-H929, U266B1, NK92MI-CD16a (ImmuneOnco Biopharmaceuticals (Shanghai) Inc.); PBS buffer; 5,6-carboxyfluorescein diacetate, succinimidyl ester, CFSE (eBioscience, 65-0850-84); propidium bromide (PI; Sigma, P4170).

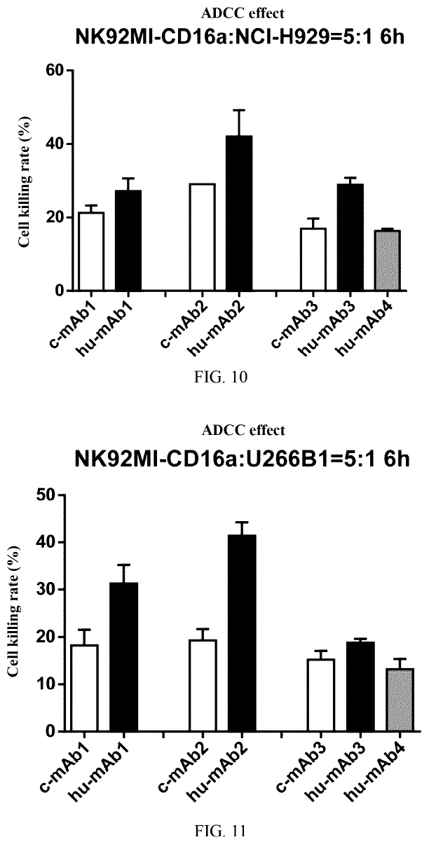

Procedures: Target cells NCI-H929 and U266B1 and effector cells NK92MI-CD16a were counted and detected for viability. The cells were centrifuged for 5 min at 300×g, stained with 5 μM CFSE (37° C., 15 min), washed twice with complete medium and counted on a VI-Cell counter (Beckman). After that, the cells were added into a 96-well plate at 2×10⁴ cells/100 μL/well according to the design. The prepared 4× antibody solutions were added at 50 μL and NK92MI-CD16a was added to the 96-well plate (1×10⁵ cells/50 μL/well, effector-to-target ratio=5:1). The cell culture plate was incubated in a cell incubator for 6 h. A PI solution with a final concentration of 1 μg/mL was added. After 10 min of incubation, the cells were detected on a flow cytometer (BD Accuri™ C6), and the percentage of CFSE+/PI+dual positive cells in CFSE+positive cells was analyzed. As shown in FIGS. 10 and 11, the chimeric antibodies c-mAb 1, c-mAb2 and c-mAb3 and the humanized antibodies hu-mAb1, hu-mAb2, hu-mAb3 and hu-mAb4 of the present invention all had significant ADCC effect on BCMA positive target cells. In addition, when NCI-H929 cells were treated with 0.1 pM antibodies in an effector-to-target ratio of 5:1, the humanized monoclonal antibodies exhibited 20%-70% improvements as compared with the parent antibodies; when U266B1 cells were treated with 100 pM antibodies in an effector-to-target ratio of 5:1, the humanized monoclonal antibodies exhibited 20%-120% improvements as compared with the parent antibodies.

1. An anti-BCMA antibody or an antigen-binding fragment thereof, comprising:
(i) an HCDR1, an HCDR2 and an HCDR3 comprised in a heavy chain variable region set forth in SEQ ID NO: 1; and/or an LCDR1, an LCDR2 and an LCDR3 comprised in a light chain variable region set forth in SEQ ID NO: 2;
(ii) an HCDR1, an HCDR2 and an HCDR3 comprised in a heavy chain variable region set forth in SEQ ID NO: 3; and/or an LCDR1, an LCDR2 and an LCDR3 comprised in a light chain variable region set forth in SEQ ID NO: 4;
(iii) an HCDR1, an HCDR2 and an HCDR3 comprised in a heavy chain variable region set forth in SEQ ID NO: 5; and/or an LCDR1, an LCDR2 and an LCDR3 comprised in a light chain variable region set forth in SEQ ID NO: 6; or
(iv) an HCDR1, an HCDR2 and an HCDR3 comprised in a heavy chain variable region; and/or an LCDR1, an LCDR2 and an LCDR3 comprised in a light chain variable region; wherein compared to the heavy chain variable region and/or the light chain variable region according to any of (i) to (iii), at least one CDR of the heavy chain variable region and/or the light chain variable region comprises a mutation that is a substitution, deletion or addition of one or more amino acids (e.g., a substitution, deletion or addition of 1, 2 or 3 amino acids) and is capable of retaining the binding affinity to BCMA; preferably, the mutation is a conservative amino acid mutation;
preferably, the CDRs are defined according to the IMGT, Kabat, Chothia or AbM numbering system; preferably, the anti-BCMA antibody comprises the following CDRs according to the IMGT numbering system,
(1) an HCDR1 comprising or consisting of an amino acid sequence set forth in SEQ ID NO: 7 or a variant thereof,
an HCDR2 comprising or consisting of an amino acid sequence set forth in SEQ ID NO: 8 or a variant thereof,
an HCDR3 comprising or consisting of an amino acid sequence set forth in SEQ ID NO: 9 or a variant thereof,
an LCDR1 comprising or consisting of an amino acid sequence set forth in SEQ ID NO: 16 or a variant thereof,
an LCDR2 comprising or consisting of an amino acid sequence set forth in SEQ ID NO: 17 or a variant thereof, and
an LCDR3 comprising or consisting of an amino acid sequence set forth in SEQ ID NO: 18 or a variant thereof;
(2) an HCDR1 comprising or consisting of an amino acid sequence set forth in SEQ ID NO: 10 or a variant thereof,
an HCDR2 comprising or consisting of an amino acid sequence set forth in SEQ ID NO: 11 or a variant thereof,
an HCDR3 comprising or consisting of an amino acid sequence set forth in SEQ ID NO: 12 or a variant thereof,
an LCDR1 comprising or consisting of an amino acid sequence set forth in SEQ ID NO: 19 or a variant thereof,
an LCDR2 comprising or consisting of an amino acid sequence set forth in SEQ ID NO: 20 or a variant thereof, and
an LCDR3 comprising or consisting of an amino acid sequence set forth in SEQ ID NO: 21 or a variant thereof; or
(3) an HCDR1 comprising or consisting of an amino acid sequence set forth in SEQ ID NO: 13 or a variant thereof,
an HCDR2 comprising or consisting of an amino acid sequence set forth in SEQ ID NO: 14 or a variant thereof,
an HCDR3 comprising or consisting of an amino acid sequence set forth in SEQ ID NO: 15 or a variant thereof,
an LCDR1 comprising or consisting of an amino acid sequence set forth in SEQ ID NO: 22 or a variant thereof,
an LCDR2 comprising or consisting of an amino acid sequence set forth in SEQ ID NO: 17 or a variant thereof, and
an LCDR3 comprising or consisting of an amino acid sequence set forth in SEQ ID NO: 23 or a variant thereof,
wherein the variant is a sequence having at least 80%, 81%, 82%, 83%, 84%, 85%, 86%, 87%, 88%, 89% or 90%, preferably at least 91%, 92%, 93%, 94%, 95%, 96%, 97%, 98% or 99% sequence identity to the corresponding CDR sequence set forth in the SEQ ID NOs and retaining the binding affinity to BCMA, or an amino acid sequence having one or more (preferably 1, 2 or 3) conservative amino acid mutations (preferably substitutions, insertions or deletions) as compared to the corresponding CDR sequence set forth in the SEQ ID NOs and retaining the binding affinity to BCMA;
preferably, the heavy chain variable region and/or the light chain variable region of the antibody or the antigen-binding fragment thereof comprises FR regions from human, mouse or rabbit.

2. The anti-BCMA antibody or the antigen-binding fragment thereof according to claim 1, wherein the heavy chain variable region of the antibody comprises or consists of a sequence set forth in SEQ ID NO: 1, SEQ ID NO: 3, SEQ ID NO: 5, SEQ ID NO: 24, SEQ ID NO: 26, SEQ ID NO: 28 or SEQ ID NO: 30, or a variant thereof; and
the light chain variable region of the antibody comprises or consists of a sequence set forth in SEQ ID NO: 2, SEQ ID NO: 4, SEQ ID NO: 6, SEQ ID NO: 25, SEQ ID NO: 27, SEQ ID NO: 29 or SEQ ID NO: 31, or a variant thereof;
wherein the variant is a sequence having at least 80%, 81%, 82%, 83%, 84%, 85%, 86%, 87%, 88%, 89% or 90%, preferably at least 91%, 92%, 93%, 94%, 95%, 96%, 97%, 98% or 99% sequence identity to the amino acid sequence of the corresponding antibody heavy chain or light chain variable region set forth in the SEQ ID NOs and retaining the binding affinity to BCMA, or an amino acid sequence having one or more (preferably 1, 2, 3, 4, 5, 6, 7, 8, 9 or 10) conservative amino acid mutations (preferably substitutions, insertions or deletions) as compared to the amino acid sequence of the corresponding antibody heavy chain or light chain variable region set forth in the SEQ ID NOs and retaining the binding affinity to BCMA.

3. The anti-BCMA antibody or the antigen-binding fragment thereof according to claim 1, wherein
the heavy chain variable region of the antibody comprises an amino acid sequence set forth in SEQ ID NO: 1 or a variant thereof, and the light chain variable region of the antibody comprises an amino acid sequence set forth in SEQ ID NO: 2 or a variant thereof;
the heavy chain variable region of the antibody comprises an amino acid sequence set forth in SEQ ID NO: 3 or a variant thereof, and the light chain variable region of the antibody comprises an amino acid sequence set forth in SEQ ID NO: 4 or a variant thereof;
the heavy chain variable region of the antibody comprises an amino acid sequence set forth in SEQ ID NO: 5 or a variant thereof, and the light chain variable region of the antibody comprises an amino acid sequence set forth in SEQ ID NO: 6 or a variant thereof;
the heavy chain variable region of the antibody comprises an amino acid sequence set forth in SEQ ID NO: 24 or a variant thereof, and the light chain variable region of the antibody comprises an amino acid sequence set forth in SEQ ID NO: 25 or a variant thereof;
the heavy chain variable region of the antibody comprises an amino acid sequence set forth in SEQ ID NO: 26 or a variant thereof, and the light chain variable region of the antibody comprises an amino acid sequence set forth in SEQ ID NO: 27 or a variant thereof;
the heavy chain variable region of the antibody comprises an amino acid sequence set forth in SEQ ID NO: 28 or a variant thereof, and the light chain variable region of the antibody comprises an amino acid sequence set forth in SEQ ID NO: 29 or a variant thereof; or
the heavy chain variable region of the antibody comprises an amino acid sequence set forth in SEQ ID NO: 30 or a variant thereof, and the light chain variable region of the antibody comprises an amino acid sequence set forth in SEQ ID NO: 31 or a variant thereof.

4. The anti-BCMA antibody or the antigen-binding fragment thereof according to claim 1, wherein the antibody comprises a heavy chain constant region and a light chain constant region that are derived from human IgG1, IgG2, IgG3 or IgG4; preferably, the heavy chain constant region is a human IgG1 heavy chain constant region, GenBank ACCESSION: AK303185.1, and the light chain constant region is an Ig1 kappa chain constant region, GenBank ACCESSION: MG815648.1.

5. The anti-BCMA antibody or the antigen-binding fragment thereof according to claim 1, wherein the antibody is a monoclonal antibody, a partially or fully humanized antibody, a chimeric antibody, a single-chain antibody and a multispecific antibody (e.g., a bispecific antibody).

6. The anti-BCMA antibody or the antigen-binding fragment thereof according to claim 1, wherein the antigen-binding fragment is selected from Fab, Fab′, F(ab′)₂, F(ab)₂, Fd, Fv, dAb, Fab/c, complementarity determining region fragment, scFv, scFv multimer, disulfide-stabilized Fv (dsFv), (dsFv)₂, bispecific dsFv (dsFv-dsFv′), diabody, disulfide-stabilized diabody (ds-diabody), multispecific antibody formed from a portion of an antibody comprising one or more CDRs, single-domain antibody (sdab), nanobody, domain antibody or bivalent domain antibody.

7. An isolated polypeptide or a variant thereof, selected from the group consisting of:
(1) an isolated polypeptide or a variant thereof, comprising sequences set forth in SEQ ID NO: 7, SEQ ID NO: 8 and SEQ ID NO: 9, wherein the polypeptide specifically binds to BCMA as part of an anti-BCMA antibody, the antibody further comprising sequences set forth in SEQ ID NO: 16, SEQ ID NO: 17 and SEQ ID NO: 18;
(2) an isolated polypeptide or a variant thereof, comprising sequences set forth in SEQ ID NO: 16, SEQ ID NO: 17 and SEQ ID NO: 18, wherein the polypeptide specifically binds to BCMA as part of an anti-BCMA antibody, the antibody further comprising sequences set forth in SEQ ID NO: 7, SEQ ID NO: 8 and SEQ ID NO: 9;
(3) an isolated polypeptide or a variant thereof, comprising sequences set forth in SEQ ID NO: 10, SEQ ID NO: 11 and SEQ ID NO: 12, wherein the polypeptide specifically binds to BCMA as part of an anti-BCMA antibody, the antibody further comprising sequences set forth in SEQ ID NO: 19, SEQ ID NO: 20 and SEQ ID NO: 21;
(4) an isolated polypeptide or a variant thereof, comprising sequences set forth in SEQ ID NO: 19, SEQ ID NO: 20 and SEQ ID NO: 21, wherein the polypeptide specifically binds to BCMA as part of an anti-BCMA antibody, the antibody further comprising sequences set forth in SEQ ID NO: 10, SEQ ID NO: 11 and SEQ ID NO: 12;
(5) an isolated polypeptide or a variant thereof, comprising sequences set forth in SEQ ID NO: 13, SEQ ID NO: 14 and SEQ ID NO: 15, wherein the polypeptide specifically binds to BCMA as part of an anti-BCMA antibody, the antibody further comprising sequences set forth in SEQ ID NO: 22, SEQ ID NO: 17 and SEQ ID NO: 23;
(6) an isolated polypeptide or a variant thereof, comprising sequences set forth in SEQ ID NO: 22, SEQ ID NO: 17 and SEQ ID NO: 23, wherein the polypeptide specifically binds to BCMA as part of an anti-BCMA antibody, the antibody further comprising sequences set forth in SEQ ID NO: 13, SEQ ID NO: 14 and SEQ ID NO: 15;
(7) an isolated polypeptide or a variant thereof, comprising a sequence set forth in SEQ ID NO: 1, wherein the polypeptide specifically binds to BCMA as part of an anti-BCMA antibody, the antibody further comprising a sequence set forth in SEQ ID NO: 2;
(8) an isolated polypeptide or a variant thereof, comprising a sequence set forth in SEQ ID NO: 2, wherein the polypeptide specifically binds to BCMA as part of an anti-BCMA antibody, the antibody further comprising a sequence set forth in SEQ ID NO: 1;
(9) an isolated polypeptide or a variant thereof, comprising a sequence set forth in SEQ ID NO: 3, wherein the polypeptide specifically binds to BCMA as part of an anti-BCMA antibody, the antibody further comprising a sequence set forth in SEQ ID NO: 4;
(10) an isolated polypeptide or a variant thereof, comprising a sequence set forth in SEQ ID NO: 4, wherein the polypeptide specifically binds to BCMA as part of an anti-BCMA antibody, the antibody further comprising a sequence set forth in SEQ ID NO: 3;
(11) an isolated polypeptide or a variant thereof, comprising a sequence set forth in SEQ ID NO: 5, wherein the polypeptide specifically binds to BCMA as part of an anti-BCMA antibody, the antibody further comprising a sequence set forth in SEQ ID NO: 6;
(12) an isolated polypeptide or a variant thereof, comprising a sequence set forth in SEQ ID NO: 6, wherein the polypeptide specifically binds to BCMA as part of an anti-BCMA antibody, the antibody further comprising a sequence set forth in SEQ ID NO: 5;
(13) an isolated polypeptide or a variant thereof, comprising a sequence set forth in SEQ ID NO: 24, wherein the polypeptide specifically binds to BCMA as part of an anti-BCMA antibody, the antibody further comprising a sequence set forth in SEQ ID NO: 25;
(14) an isolated polypeptide or a variant thereof, comprising a sequence set forth in SEQ ID NO: 25, wherein the polypeptide specifically binds to BCMA as part of an anti-BCMA antibody, the antibody further comprising a sequence set forth in SEQ ID NO: 24;
(15) an isolated polypeptide or a variant thereof, comprising a sequence set forth in SEQ ID NO: 26, wherein the polypeptide specifically binds to BCMA as part of an anti-BCMA antibody, the antibody further comprising a sequence set forth in SEQ ID NO: 27;
(16) an isolated polypeptide or a variant thereof, comprising a sequence set forth in SEQ ID NO: 27, wherein the polypeptide specifically binds to BCMA as part of an anti-BCMA antibody, the antibody further comprising a sequence set forth in SEQ ID NO: 26;
(17) an isolated polypeptide or a variant thereof, comprising a sequence set forth in SEQ ID NO: 28, wherein the polypeptide specifically binds to BCMA as part of an anti-BCMA antibody, the antibody further comprising a sequence set forth in SEQ ID NO: 29;
(18) an isolated polypeptide or a variant thereof, comprising a sequence set forth in SEQ ID NO: 29, wherein the polypeptide specifically binds to BCMA as part of an anti-BCMA antibody, the antibody further comprising a sequence set forth in SEQ ID NO: 28;
(19) an isolated polypeptide or a variant thereof, comprising a sequence set forth in SEQ ID NO: 30, wherein the polypeptide specifically binds to BCMA as part of an anti-BCMA antibody, the antibody further comprising a sequence set forth in SEQ ID NO: 31;
or
(20) an isolated polypeptide or a variant thereof, comprising a sequence set forth in SEQ ID NO: 31, wherein the polypeptide specifically binds to BCMA as part of an anti-BCMA antibody, the antibody further comprising a sequence set forth in SEQ ID NO: 30,
wherein the variant is a sequence having at least 80%, 81%, 82%, 83%, 84%, 85%, 86%, 87%, 88%, 89% or 90%, preferably at least 91%, 92%, 93%, 94%, 95%, 96%, 97%, 98% or 99% sequence identity to the corresponding sequence set forth in the SEQ ID NOs and retaining the binding affinity to BCMA, or an amino acid sequence having one or more (preferably 1, 2, 3, 4, 5, 6, 7, 8, 9 or 10) conservative amino acid mutations (preferably substitutions, insertions or deletions) as compared to the corresponding sequence set forth in the SEQ ID NOs and retaining the binding affinity to BCMA.

8. A biological material selected from the group consisting of the followings:
(1) a nucleic acid molecule encoding the antibody or the antigen-binding fragment thereof according to claim 1;
(2) a vector comprising the nucleic acid molecule according to item (1); and
(3) a host cell comprising the nucleic acid molecule according to item (1) or the vector according to item (2).

9. A biological material selected from the group consisting of the followings:
(1) a nucleic acid molecule encoding the isolated polypeptide or the variant thereof according to claim 7;
(2) a vector comprising the nucleic acid molecule according to item (1); and
(3) a host cell comprising the nucleic acid molecule according to item (1) or the vector according to item (2).

10. (canceled)

11. A product selected from the group consisting of the followings:
(1) a conjugate comprising the antibody or the antigen-binding fragment thereof according to claim 1 and a conjugated moiety, wherein the conjugated moiety is a purification tag (e.g., His tag), a detectable label, a drug, a toxin, a cytokine, an enzyme or a combination thereof; preferably, the conjugated moiety is a radioisotope, a fluorescent substance, a chemiluminescent substance, a colored substance, a chemotherapeutic agent, a biotoxin, polyethylene glycol or an enzyme;
(2) a fusion protein or a multispecific antibody (preferably a bispecific antibody) comprising the antibody or the antigen-binding fragment thereof according to claim 1, wherein preferably, the fusion protein is a CAR construct that specifically binds to BCMA;
(3) a kit comprising the antibody or the antigen-binding fragment thereof according to claim 1, the conjugate according to item (1), or the fusion protein or the multispecific antibody according to item (2), wherein preferably, the kit further comprises a second antibody that specifically recognizes the antibody; optionally, the second antibody further comprises a detectable label, such as a radioisotope, a fluorescent substance, a chemiluminescent substance, a colored substance or an enzyme; preferably, the kit is configured for detecting the presence or level of BCMA in a sample;
(4) a pharmaceutical composition comprising the antibody or the antigen-binding fragment thereof according to claim 1, the conjugate according to item (1), or the fusion protein or the multispecific antibody according to item (2), wherein optionally, the pharmaceutical composition further comprises a pharmaceutically acceptable carrier and/or excipient preferably, the pharmaceutical composition is in a form suitable for administration through subcutaneous injection, intradermal injection, intravenous injection, intramuscular injection or intralesional injection; and
(5) a kit comprising
(a) the antibody or the antigen-binding fragment thereof according to claim 1, the conjugate according to item (1), or the fusion protein or the multispecific antibody according to item (2), and
(b) an antibody or an antigen-binding fragment thereof against another antigen (e.g., CD38 and/or SLAMF7), and/or a cytotoxic agent, and optionally, a package insert.

12-16. (canceled)

17. A method for treating or preventing a tumor (e.g., multiple myeloma) comprising administering to a subject a therapeutically effective amount of the antibody or the antigen-binding fragment thereof according to claim 1.

18. A method for treating or preventing a tumor comprising administering to a subject a therapeutically effective amount of the conjugate according to claim 11.

19. A method for treating or preventing a tumor comprising administering to a subject a therapeutically effective amount of the fusion protein or the multispecific antibody according to claim 11.


SEQUENCE LISTING (36 sequences)

SEQ ID NO: 1 - protein, 119 aa, Artificial Sequence
  Synthetic Artificial Sequence
EVQLQQSGPELVKPGTSVEMSCKASGYTFTDYNIHWVRQRHGESLEWLGYINPNNGVTNFNQKFKGKATLTVNKSSSTAYMELRSLTSDDSAVYYCSSGLFDGYPFPYWGQGTLVTVSA

SEQ ID NO: 2 - protein, 111 aa, Artificial Sequence
  Synthetic Artificial Sequence
DIVLTQSPASLVVSLGQRATISCRASKSVSTSGYNYIHWYQQKPGQPPKLLIFLASNLKSGVPARFSGGGSGTDFTLNIHPVEEDDAATYFCQHNRELPYTFGGGTKVEIK

SEQ ID NO: 3 - protein, 118 aa, Artificial Sequence
  Synthetic Artificial Sequence
QVQLQQSDAELVKPGTSVKISCKVSGYTFTDHTIHWMKQRPEQGLEWIGYIYPRNGNTKYNEKFKGKATLTADKSSSTAYMHLNSLTSEDSAVYFCARYDYDGYFDVWGTGTTVTVSS

SEQ ID NO: 4 - protein, 112 aa, Artificial Sequence
  Synthetic Artificial Sequence
DVVMTQTPLSLPVSLGDQASISCRSSQSHVHSDGHTYLHWYLQKPGQSPELLIYKVSNRFSGVPDRFSGSGSGTDFTLKISRVEAEDLGVYFCSQNTHFPWTFGGGTKLEIK

SEQ ID NO: 5 - protein, 118 aa, Artificial Sequence
  Synthetic Artificial Sequence
EVQLQQSGPELVKPGASVKILCKASGYTITDYNMDWVKRSHGKSLEWIGNINPHNGGSIYNQKFKGKATLTVDKSSSTAFMELRSLTSEDTAVYYCARGDATLVLDYWGQGTSLTVSS

SEQ ID NO: 6 - protein, 111 aa, Artificial Sequence
  Synthetic Artificial Sequence
DIVLTQSPASLAVSLGQRATISCRASKSVSTSGLSYMHWYQQKPGQPPKVLIYLASNLESGVPARFSGSGSGTDFTLNIHPVEEEDAATYYCQHSRELPPTFGAGTKLELK

SEQ ID NO: 7 - protein, 8 aa, Artificial Sequence
  Synthetic Artificial Sequence
GYTFTDYN

SEQ ID NO: 8 - protein, 8 aa, Artificial Sequence
  Synthetic Artificial Sequence
INPNNGVT

SEQ ID NO: 9 - protein, 14 aa, Artificial Sequence
  Synthetic Artificial Sequence
CSSGLFDGYPFPYW

SEQ ID NO: 10 - protein, 8 aa, Artificial Sequence
  Synthetic Artificial Sequence
GYTFTDHT

SEQ ID NO: 11 - protein, 8 aa, Artificial Sequence
  Synthetic Artificial Sequence
IYPRNGNT

SEQ ID NO: 12 - protein, 13 aa, Artificial Sequence
  Synthetic Artificial Sequence
CARYDYDGYFDVW

SEQ ID NO: 13 - protein, 8 aa, Artificial Sequence
  Synthetic Artificial Sequence
GYTITDYN

SEQ ID NO: 14 - protein, 8 aa, Artificial Sequence
  Synthetic Artificial Sequence
INPHNGGS

SEQ ID NO: 15 - protein, 13 aa, Artificial Sequence
  Synthetic Artificial Sequence
CARGDATLVLDYW

SEQ ID NO: 16 - protein, 10 aa, Artificial Sequence
  Synthetic Artificial Sequence
KSVSTSGYNY

SEQ ID NO: 17 - protein, 3 aa, Artificial Sequence
  Synthetic Artificial Sequence
LAS

SEQ ID NO: 18 - protein, 11 aa, Artificial Sequence
  Synthetic Artificial Sequence
CQHNRELPYTF

SEQ ID NO: 19 - protein, 11 aa, Artificial Sequence
  Synthetic Artificial Sequence
QSHVHSDGHTY

SEQ ID NO: 20 - protein, 3 aa, Artificial Sequence
  Synthetic Artificial Sequence
KVS

SEQ ID NO: 21 - protein, 11 aa, Artificial Sequence
  Synthetic Artificial Sequence
CSQNTHFPWTF

SEQ ID NO: 22 - protein, 10 aa, Artificial Sequence
  Synthetic Artificial Sequence
KSVSTSGLSY

SEQ ID NO: 23 - protein, 11 aa, Artificial Sequence
  Synthetic Artificial Sequence
CQHSRELPPTF

SEQ ID NO: 24 - protein, 111 aa, Artificial Sequence
  Synthetic Artificial Sequence
DIVLTQSPDSLAVSLGREATINCRASKSVSTSGYNYIHWYQQKPGQPPKLLIFLASNLKSGVPDRFSGSGSGTDFTLTISSLQAEDVAVYYCQHNRELPYTFGGGTKVEIK

SEQ ID NO: 25 - protein, 118 aa, Artificial Sequence
  Synthetic Artificial Sequence
EVQLVESGGALVKPGGSLRLSCKASGYTITDYNMDWVRQSPGKGLEWIGNINPHNGGSIYNQKFQGRATITVDKSTSTAYMELSSLRSEDTAVYYCARGDATLVLDYWGQGTLVTVSS

SEQ ID NO: 26 - protein, 118 aa, Artificial Sequence
  Synthetic Artificial Sequence
QVQLVQSGAEVKKPGSSVKVSCKVSGYTFTDHTIHWMKQAPGQGLEWIGYIYPRNGNTKYNEKFKGKATLTADKSTSTAYMELSSLRSEDTAVYFCARYDYDGYFDVWGQGTLVTVSS

SEQ ID NO: 27 - protein, 112 aa, Artificial Sequence
  Synthetic Artificial Sequence
DVVMTQTPLSLSVTPGQPASISCRSSQSHVHSDGHTYLHWYLQKPGQSPELLIYKVSNRFSGVPDRFSGSGSGTDFTLKISRVEAEDVGVYFCSQNTHFPWTFGGGTKVEIK

SEQ ID NO: 28 - protein, 118 aa, Artificial Sequence
  Synthetic Artificial Sequence
QVQLVQSGAEVKKPGASVKVSCKASGYTITDYNMDWVRQAPGQGLEWIGNINPHNGGSIYNQKFQGRATLTVDKSTSTAYMELSSLRSEDTAVYYCARGDATLVLDYWGQGTTVTVSS

SEQ ID NO: 29 - protein, 111 aa, Artificial Sequence
  Synthetic Artificial Sequence
DIQLTQSPSSLSASVGDRATITCRASKSVSTSGLSYMHWYQQKPGKAPKVLIYLASNLESGVPARFSGSGSGTDFTLTISSVQPEDFATYYCQHSRELPPTFGQGTKLELK

SEQ ID NO: 30 - protein, 119 aa, Artificial Sequence
  Synthetic Artificial Sequence
EVQLVQSGAEVKKPGASVKVSCKASGYTFTDYNIHWVRQAPGQGLEWLGYINPNNGVTNFNQKFKGRATLTVDKSTSTAYMELRSLRSDDTAVYYCSSGLFDGYPFPYWGQGTLVTVSS

SEQ ID NO: 31 - protein, 111 aa, Artificial Sequence
  Synthetic Artificial Sequence
EIVLTQSPATLSVSPGREATLSCRASKSVSTSGLSYMHWYQQKPGQAPRVLIYLASNLESGVPARFSGSGSGTEFTLTISSLQSEDAAVYYCQHSRELPPTFGQGTKLEIK

SEQ ID NO: 32 - protein, 54 aa, Artificial Sequence
  Synthetic Artificial Sequence
MLQMAGQCSQNEYFDSLLHACIPCQLRCSSNTPPLTCQRYCNASVTNSVKGTNA

SEQ ID NO: 33 - protein, 52 aa, Artificial Sequence
  Synthetic Artificial Sequence
MLQMARQCSQNEYFDSLLHDCKPCQLRCSSTPPLTCQRYCNASMTNSVKGMN

SEQ ID NO: 34 - protein, 49 aa, Artificial Sequence
  Synthetic Artificial Sequence
MAQQCFHSEYFDSLLHACKPCHLRCSNPPATCQPYCDPSVTSSVKGTYT

SEQ ID NO: 35 - protein, 237 aa, Artificial Sequence
  Synthetic Artificial Sequence
IEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 233 aa, Artificial Sequence
  Synthetic Artificial Sequence
EPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK